SEQUENCE LISTING (17 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 200 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 5 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 6 - protein, 8 aa, Homo sapiens
PQGHSDTT

SEQ ID NO: 7 - protein, 7 aa, Homo sapiens
PQGHSDT

SEQ ID NO: 8 - protein, 6 aa, Homo sapiens
PQGHSD

SEQ ID NO: 9 - protein, 5 aa, Homo sapiens
PQGHS

SEQ ID NO: 10 - protein, 4 aa, Homo sapiens
PQGH

SEQ ID NO: 11 - protein, 3 aa, Homo sapiens
PQG

SEQ ID NO: 12 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 13 - protein, 19 aa, Homo sapiens
NWELTASASHQPPGVYPQG

SEQ ID NO: 14 - protein, 17 aa, Homo sapiens
ITCPPPMSVEHADIWVK

SEQ ID NO: 15 - protein, 31 aa, Homo sapiens
ITCPPPMSVEHADIWVKSYSLYSRERYICNS

SEQ ID NO: 16 - protein, 436 aa, Artificial Sequence
  Description of Artificial Sequence Synthetic polypeptide
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 431 aa, Artificial Sequence
  Description of Artificial Sequence Synthetic polypeptide
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0330747 A1
(43) Pub. Date: Oct. 28, 2021

(54) Pharmaceutical Compositions and Pharmaceutical Products of Heterodimeric Human Interleukin-15 (hetIL-15)

(71) Applicant: Novartis AG, Basel (CH)

(72) Inventors: Ingo FISCHER, Allschwil (CH); Richard GABRIEL, Sierentz (FR)

(21) Appl. No.: 17/234,914

(22) Filed: Apr. 20, 2021

Related U.S. Application Data
(60) Provisional application No. 63/013,801, filed on Apr. 22, 2020.

(51) Int. Cl. A61K 38/20; A61K 38/17; A61K 47/10; A61K 47/26

(52) U.S. Cl. CPC A61K 38/2086; A61K 47/26; A61K 47/10; A61K 38/1793

(57) ABSTRACT

The disclosure is directed to stable pharmaceutical compositions comprising a heterodimer complex of IL-15 and IL-15Rα and pharmaceutical products comprising such compositions. The disclosure is also directed to the use of these compositions (e.g. as part of a kit having instructions for use) and pharmaceutical products for the treatment of lymphopenia, cancer, or infectious disease.

### CROSS REFERENCE TO RELATED APPLICATIONS

This application claims the benefit of U.S. Provisional Application No. 63/013,801 filed on Apr. 22, 2020, which is hereby incorporated by reference in its entirety.

### FIELD OF THE DISCLOSURE

The disclosure is directed to pharmaceutical compositions of heterodimeric human interleukin-15 (IL-15/IL-15Rα) complex, pharmaceutical products comprising such pharmaceutical compositions, and uses of the pharmaceutical compositions. In particular, the disclosure concerns stable liquid and solid pharmaceutical formulations comprising a heterodimeric IL-15/IL-15Rα complex, e.g. as disclosed herein.

### SEQUENCE LISTING

The instant application contains a Sequence Listing which has been submitted electronically in ASCII format and is hereby incorporated by reference in its entirety. Said ASCII copy, created on Mar. 31, 2021, is named PAT058681-SL.txt and is 19,095 bytes in size

### BACKGROUND OF THE DISCLOSURE

The cytokine interleukin-15 (IL-15) is a member of the four alpha-helix bundle family of lymphokines produced by many cells in the body. IL-15 plays a pivotal role in modulating the activity of both the innate and adaptive immune system, e.g. maintenance of the memory T-cell response to invading pathogens, inhibition of apoptosis, activation of dendritic cells, and induction of Natural Killer (NK) cell proliferation and cytotoxic activity.

The IL-15 receptor consists of three polypeptides, the type-specific IL-15 receptor alpha (“IL-15Rα”), the IL-2/IL-15 receptor beta (or CD122) (“β”), and the common gamma chain (or CD132) (“γ”) that is shared by multiple cytokine receptors. IL-15Rα is thought to be expressed by a wide variety of cell types, but not necessarily in conjunction with β and γ. IL-15 signaling has been shown to occur through the heterodimeric complex of IL-15Rα, β, and γ; through the heterodimeric complex of β and γ, or through a subunit, IL-15RX, found on mast cells.

IL-15 specifically binds to the IL-15Rα with high affinity via the “sushi domain” in exon 2 of the extracellular domain of the receptor. After trans-endosomal recycling and migration back to the cell surface, these IL-15 complexes acquire the property to activate bystander cells expressing the IL-15R βγ low-affinity receptor complex, inducing IL-15-mediated signaling via the Jak/Stat pathway. A wild-type soluble form of IL-15Rα (“sIL-15Rα”), which is cleaved at a cleavage site in the extracellular domain immediately distal to the transmembrane domain of the receptor has been observed.

Based on its multifaceted role in the immune system, various therapies designed to modulate IL-15-mediated function have been explored. Recent reports suggest that IL-15, when complexed with the sIL-15Rα, or the sushi domain, maintains its immune enhancing function. Recombinant IL-15 and IL-15/IL-15Rα complexes have been shown to promote to different degrees the expansion of memory CD8 T cells and NK cells and enhance tumor rejection in various preclinical models. Furthermore, tumor targeting of IL-15 or IL-15/IL-15Rα complex containing constructs in mouse models, resulted in improved anti-tumor responses in either immunocompetent animals transplanted with syngeneic tumors or in T- and B cell-deficient SCID mice (retaining NK cells) injected with human tumor cell lines.

Therapeutic proteins are typically formulated either in aqueous form ready for parenteral administration or as lyophilizates for reconstitution with a suitable diluent prior to administration.

Therapeutic proteins in lyophilizates are stable over long periods of time and can be reconstituted to give a solution of the active ingredient. It is desirable that the reconstituted solution has a low level of protein aggregation for delivery to a patient.

Pharmaceutical compositions have short shelf lives and the formulated proteins may lose biological activity resulting from chemical and physical instabilities during storage. Pharmaceutical products comprising proteins are very susceptible to physical and chemical degradation and the marginal stability of proteins in liquid compositions often prevents long-term storage at room temperature or refrigerated conditions, while lyophilizates are generally more stable. Physical and chemical reactions can occur in solution (aggregation [covalent and noncovalent], deamidation, oxidation, clipping, isomerization, denaturation), leading to an increase in degradation product levels and/or loss of bioactivity.

A composition comprising a protein or protein complex, e.g. IL-15/IL-15Rα complex, should provide sufficient physical and chemical stability of the protein or protein complex, e.g. IL-15/IL-15Rα complex, during shipping and handling to ensure that the dosage and product safety claims are met when the molecule is administered to a patient. Specifically, an acceptable composition comprising protein or protein complex, e.g. IL-15/IL-15Rα complex, must enhance stability and minimize protein degradation, especially protein aggregation, in order to avoid serious immunogenic reactions and retain a biologically active molecule. Moreover, the composition must also be of acceptable osmolality and pH value for subcutaneous application and have low viscosity as a prerequisite for manufacturing (compounding, filtration, filling) and syringeability. However, a long appreciated problem with pharmaceutical formulations of protein therapeutics is that of stability and aggregation, where protein molecules stick together, and can results in formation of opaque insoluble matter or precipitation, which may block syringes or pumps or which may show undesired reactions after administration, rendering it unsafe for patients.

### SUMMARY OF THE DISCLOSURE

Presently provided are pharmaceutical compositions comprising heterodimeric IL-15/IL-15Rα complex, e.g. as disclosed herein, which are stable for extended periods of time. Such pharmaceutical compositions can be solid compositions or liquid compositions.

In one aspect, disclosed herein are liquid pharmaceutical compositions comprising a heterodimeric IL-15/IL-15Rα complex, e.g. as disclosed herein, and about 0.0001% to about 1% (w/v) of a surfactant, optionally further comprising about 1 mM to about 100 mM of a buffering agent providing a pH in the range of from about 4.5 to about 8.5, optionally further comprising about 1 mM to about 500 mM of at least one stabilizer as well as subcombinations thereof. The liquid composition is not reconstituted from a lyophilizate, but rather is a ready-to-use liquid composition.

In one aspect, disclosed herein are liquid pharmaceutical compositions comprising a heterodimeric IL-15/IL-15Rα complex, e.g. as disclosed herein, and no surfactant, optionally further comprising about 1 mM to about 100 mM of a buffering agent providing a pH in the range of from about 4.5 to about 8.5, optionally further comprising about 1 mM to about 500 mM of at least one stabilizer as well as subcombinations thereof. The liquid composition is not reconstituted from a lyophilizate, but rather is a ready-to-use liquid composition.

Also disclosed herein are pharmaceutical products comprising: a container and a liquid pharmaceutical composition disposed within said container, said composition comprising about 0.1 mg/mL to about 50 mg/mL or about 0.1 mg/mL to about 10 mg/mL of a heterodimeric IL-15/IL-15Rα complex, e.g. as disclosed herein, and optionally about 0.0001% to about 1% (w/v) of a surfactant, optionally further comprising about 1 mM to about 100 mM of a buffering agent providing a pH in the range of from about 4.5 to about 8.5, optionally further comprising about 1 mM to about 500 mM of at least one stabilizer as well as subcombinations thereof, wherein the liquid pharmaceutical composition is not reconstituted from a lyophilizate.

In another aspect, disclosed herein are solid pharmaceutical compositions comprising a heterodimeric IL-15/IL-15Rα complex; and about 1 mM to about 100 mM of a buffering agent providing a pH in the range of from about 6.5 to about 8.5, and about 1 mM to about 500 mM of at least one stabilizer as well as subcombinations thereof.

Also disclosed herein are pharmaceutical solid products comprising: a container and a pharmaceutical composition disposed within said container, said composition comprising about 0.1 mg/mL to about 50 mg/mL or about 0.1 mg/mL to about 10 mg/mL of a heterodimeric IL-15/IL-15Rα complex, e.g. as disclosed herein; comprising about 1 mM to 100 mM of a buffering agent providing a pH in the range of from about 6.5 to about 8.5 and about 1 mM to about 500 mM of at least one stabilizer as well as sub-combinations thereof.

The disclosure is also directed to the use of these pharmaceutical compositions for the treatment of lymphopenia, cancer, or infectious disease and to kits containing these pharmaceutical products and compositions.

Additional compositions, products, methods, regimens, uses, and kits are provided in the following description and appended claims.

### BRIEF DESCRIPTION OF THE DRAWINGS

FIGS. 1A-C: Sum of aggregates of formulations F1 to F3 following storage at A) 2-8° C. for 6 months (24 weeks), B) at 25° C. for 3 months (12 weeks) and C) 40° C. for 1.5 months (6 weeks).

FIGS. 2A-C: Sum of degradation products by SEC for formulations F1 to F3 following storage at A) 2-8° C. for 6 months (24 weeks), B) at 25° C. for 3 months (12 weeks) and C) 40° C. for 1.5 months (6 weeks).

FIGS. 3A-B: Sum of charge variants for formulations F1 to F3 following storage at A) 2-8° C. for 6 months (24 weeks) and B) at 25° C. for 3 months (12 weeks).

FIGS. 4A-D: Purity by CE-SDS for A) IL-15 receptor alpha (IL-15Rα), B) IL-15 main species, C) IL-15 high molecular weight species (HMW) and D) aglycosylated IL-15 in formulations F1 to F3 following storage at 2-8° C. for 6 months (24 weeks).

FIGS. 5A-C: Purity by RP-HPLC for formulations F1 to F3 following storage at A) 2-8° C. for 6 months (24 weeks), B) at 25° C. for 3 months (12 weeks) and C) 40° C. for 1.5 months (6 weeks).

FIGS. 6A-B: Number of subvisible particles (SVP) assessed by PAMAS A) greater than 2 pm in size and B) greater than 10 μm in size in formulations F1 to F3 following storage at 2-8° C. for 6 months (24 weeks).

FIG. 7: Turbidity of formulations F1 to F3 following storage at 2-8° C. for 6 months (24 weeks) (NTU=Nephelometric Turbidity Units).

FIGS. 8A-B: Mechanical stress results for F2 and F3 subjected to five freeze/thaw cycles or overnight shaking. Shown are A) sum of aggregates and B) sum of fragments as assessed by SEC.

FIGS. 9A-E: Number of subvisible particles (SVP) assessed by PAMAS A) greater than 2 μm in size and B) greater than 10 μm in size in the formulation comprising acetate at pH 4.7 to 5.5 in the presence of polysorbate 20 or poloxamer 188 following storage at 2-8° C. for 5 months (SVP>2 μm) and 4 months (SVP>10 μm), respectively. Number of subvisible particles (SVP) assessed by PAMAS C) greater than 2 μm in size , D) greater than 5 μm in size and E) greater than 10 μm in size for all formulations following storage at 2-8° C. up to 12 months

FIG. 10: Purity by RP-HPLC for all formulations following storage at 40° C. up to 3 months.

### DETAILED DESCRIPTION OF THE DISCLOSURE

The invention relates to a pharmaceutical formulation comprising heterodimeric IL-15/IL-15Rα complex, e.g. as disclosed herein. The pharmaceutical formulation may be in a solid (e.g. lyophilized) or liquid form.

In order that the disclosure may be more readily understood, certain terms are defined below and throughout the detailed description. Unless defined otherwise herein, all scientific and technical terms used in connection with the present disclosure have the same meaning as commonly understood by those of ordinary skill in the art. All publications, patent applications, patents, scientific publications and other references mentioned herein are incorporated by reference in their entirety. To the extent a cited reference conflicts with the disclosure herein, the specification shall control. Throughout the text of this application, should there be a discrepancy between the text of the specification (e.g. Table 1) and the sequence listing, the text of the specification shall prevail. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

The details of one or more aspects and embodiments of the invention are set forth in the accompanying drawings and the description below. Other features, objects, and advantages of the invention will be apparent from the description and drawings, and from the claims.

### Definitions

As used in the specification and claims, the singular form “a”, “an” and “the” refer to one or to more than one (e.g. to at least one) of the grammatical object of the article, unless the context clearly dictates otherwise. For example, the term “a cell” includes a plurality of cells, including mixtures thereof.

Throughout this specification and the claims which follow, unless the context requires otherwise, the word “comprise”, and variations such as “comprises” and “comprising”, are used herein in their open-ended and non-limiting sense unless otherwise noted. As used herein, the term “comprising” encompasses “including” as well as “consisting of” e.g. a composition “comprising” X may consist exclusively of X or may include something additional, e.g. X+Y.

When used herein “consisting of” excludes any element, step, or ingredient not specified in the aspect, embodiment and/or claim element. When used herein, “consisting essentially of” does not exclude materials or steps that do not materially affect the basic and novel characteristics of the aspect, embodiment and/or claim.

In each instance herein any one of the terms “comprising”, “consisting essentially of” and “consisting of” may be replaced with either of the other two terms.

The term “or” is used herein to mean, and is used interchangeably with, the term “and/or”, unless context clearly indicates otherwise.

All numerical designations, e.g. pH, temperature, time, concentration, and molecular weight, including ranges, are approximations which are varied (+) or (−) by increments of 0.1. It is to be understood, although not always explicitly stated that all numerical designations are preceded by the term “about”. The terms “about” and “approximately” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10% 9%, 8%, 7%, 6%, 5%, 4%, 3%, 2% or 1% from that value. In some cases, the numerical value disclosed throughout can be “about” that numerical value even without specifically mentioning the term “about” or “approximately”. It also is to be understood, although not always explicitly stated, that the reagents described herein are merely examples and that equivalents of such are known in the art.

The compositions, methods and uses described herein encompass polypeptides and nucleic acids having the sequences specified, or sequences substantially identical or similar thereto, e.g. sequences at least about 85%, at least about 90%, at least about 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, at least about 90%, at least about 91%, at least about 92%, at least about 93%, at least about 94%, at least about 95%, at least about 96%, at least about 97%, at least about 98% or at least about 99% identity to a reference sequence, e.g. a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, at least about 90%, at least about 91° A, at least about 92%, at least about 93%, at least about 94%, at least about 95%, at least about 96%, at least about 97%, at least about 98% or at least about 99% identity to a reference sequence, e.g. a sequence provided herein.

The term “functional variant” refers to polypeptides that have a substantially identical amino acid sequence to the naturally-occurring or wild type sequence, or are encoded by a substantially identical nucleotide sequence, and are capable of having one or more activities of the naturally-occurring or wild type sequence.

To determine the percent identity of two amino acid sequences, or of two nucleic acid sequences, the sequences are aligned for optimal comparison purposes (e.g. gaps can be introduced in one or both of a first and a second amino acid or nucleic acid sequence for optimal alignment and non-homologous sequences can be disregarded for comparison purposes). Suitably, the length of a reference sequence aligned for comparison purposes is at least 70%, at least 80%, at least 90%, at least 95%, or at least 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences is determined using the Needleman and Wunsch ((1970) J. Mol. Biol. 48:444-453) algorithm which has been incorporated into the GAP program in the GCG software package (available from the NCBI), using either a Blossum 62 matrix or a PAM250 matrix, and a gap weight of 16, 14, 12, 10, 8, 6, or 4 and a length weight of 1, 2, 3, 4, 5, or 6. Suitably, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST protein searches can be performed with the XBLAST program, score =50, word length =3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g. XBLAST and NBLAST) can be used (available from the NBCI).

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidom imetics.

A “conservative amino acid substitution” is one in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid”, “nucleic acid sequence”, “nucleotide sequence”, “polynucleotide sequence” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “glycosylation” refers to the attachment of a polysaccharide to a polypeptide. Preferably, the polysaccharide consists of 2-12 monosaccharides linked together by glycosidic bonds. Glycoproteins can contain O-linked sugar moieties and/or N-linked sugar moieties. The structure and number of sugar moieties attached to a particular glycosylation site can be variable. Such sugar moieties may be, for instance, N-acetyl glucosamine, N-acetyl galactosamine, mannose, galactose, glucose, fucose, xylose, glucuronic acid, iduronic acid and/or sialic acids.

The term “N-linked glycosylation” refers to the attachment of a polysaccharide to an asparagine residue of an amino acid chain.

The term “O-linked glycosylation” refers to the attachment of a carbohydrate moiety to a serine or threonine residue of an amino acid chain.

The terms “sugar profile” or “glycosylation profile”, are used and describe the glycan nature of a glycosylated polypeptide. These properties are suitably the glycosylation site, or the occupancy of the glycosylation site, or the identity, structure, composition or amount of the glycan and/or non-sugar portion of the polypeptide, or the identity and amount of a specific glycoform.

As used herein, the term “glycan” is a sugar, which can be monomers or polymers of sugar residues, such as at least three sugars, and can be linear or branched (e.g. have an α 1,3 arm and an α 1,6 arm). A “glycan” can include natural sugar residues (e.g. glucose, N-acetylglucosamine, N-acetyl neuraminic acid, galactose, mannose, fucose, hexose, arabinose, ribose, xylose, etc.) and/or modified sugars (e.g. 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g. of a glycoprotein, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e. glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties. The polysaccharide is attached either via the OH group of serine or threonine (O-glycosylated polypeptide) or via the amide group (NH₂) of asparagine (N-glycosylated polypeptide). The glycoprotein may be homologous to the host cell or preferably heterologous to the host cell expressing it, i.e. foreign, e.g. a human protein produced by CHO cells.

The term “glycoconjugate” as used herein, encompasses all molecules in which at least one sugar moiety is covalently linked to at least one other moiety. The term specifically encompasses all biomolecules with covalently attached sugar moieties, including for example N-linked glycoproteins, O-linked glycoproteins, glycolipids, proteoglycans, etc.

As used herein, the term “glycosylation pattern” refers to the set of glycan structures present on a particular sample. For example, a particular glycoconjugate (e.g. glycoprotein) or set of glycoconjugates (e.g. set of glycoproteins) will have a glycosylation pattern. In some embodiments, reference is made to the glycosylation pattern of cell surface glycans. A glycosylation pattern can be characterized by, for example, the identities of glycans, amounts (absolute or relative) of individual glycans or glycans of particular types, degree of occupancy of glycosylation sites, etc., or combinations of such parameters.

As used herein, the terms “specifically binds”, “specifically recognizes” and analogous terms in the context of a receptor (e.g. native IL-15Rα or IL-15 receptor βγ) and a ligand (e.g. native IL-15) interaction refer to the specific binding or association between the ligand and receptor. Preferably, the ligand has higher affinity for the receptor than for other molecules. In a specific embodiment, the ligand is native IL-15 and the native receptor is IL-15Rα. In another specific embodiment, the ligand is the native IL-15/IL-15Rα complex and the native receptor is the βγ receptor complex. In a further embodiment, the IL-15/IL-15Rα complex binds to the βγ receptor complex and activates IL-15 mediated signal transduction. Ligands that specifically bind a receptor can be identified, for example, by immunoassays, surface plasmon resonance, e.g. BIAcore, or other techniques known to those of skill in the art.

As used herein, the terms “purified ” and “isolated” when used in the context of a compound or agent (including proteinaceous agents such as polypeptides) that can be obtained from a natural source, e.g. cells, refers to a compound or agent which is substantially free of contaminating materials from the natural source, e.g. soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g. cellular components of the cells) from which it is isolated. Thus, a compound or agent that is isolated includes preparations of a compound or agent having less than about 30%>, 20%>, 10%>, 5%, 2% or 1% (by dry weight) of cellular materials and/or contaminating materials. IL-15

As used herein, the terms “IL-15” and “interleukin-15” refer to a native IL-15 or an IL-15 derivative. As used herein, the terms “native IL-15” and “native interleukin-15” in the context of proteins or polypeptides refer to any naturally occurring and wild type mammalian interleukin-15 amino acid sequences, including immature or precursor and mature forms. Non-limiting examples of GeneBank Accession Nos. for the amino acid sequence of various species of native mammalian interleukin-15 include NP_000576 (human, immature form), CAA62616 (human, immature form), NP_001009207 (Felis catus, immature form), AAB94536 (Rattus norvegicus, immature form), AAB41697 (Rattus norvegicus, immature form), NP_032383 (Mus musculus, immature form), AAR19080 (canine), AAB60398 (Macaca mulatta, immature form), AAI00964 (human, immature form), AAH23698 (Mus musculus, immature form), and AAH18149 (human). The amino acid sequence of the immature/precursor form of native human IL-15, which comprises the long signal peptide (underlined) and the mature human native IL-15 (italicized), as provided in SEQ ID NO: 1 in Table 1. In some embodiments, native IL-15 is the immature or precursor form of a naturally occurring or wild type mammalian IL-15. In other embodiments, native IL-15 is the mature form of a naturally occurring or wild type mammalian IL-15. In a specific embodiment, native IL-15 is the precursor form of naturally occurring or wild type human IL-15. In another embodiment, native IL-15 is the mature form of naturally occurring or wild type human IL-15. In one embodiment, the native IL-15 protein/polypeptide is isolated or purified.

In a particular embodiment, the mature human IL-15 comprises the amino acid sequence of

(SEQ ID NO: 2)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS.

As used herein, the terms “IL-15 derivative” and “interleukin-15 derivative” in the context of proteins or polypeptides refer to: (a) a polypeptide that is at least 75%, at least 80%, at least 85%, at least 90%, at least 95%, at least 98% or at least 99% identical to a native mammalian IL-15 polypeptide; (b) a polypeptide that contains 1, 2, 3, 4, 5, 6, 7, 8, 9, 10, 11, 12, 13, 14, 15, 16, 17, 18, 19, 20 or more amino acid mutations (i.e. additions, deletions and/or substitutions) relative to a native mammalian IL-15 polypeptide; and/or (c) a fragment of a native mammalian IL-15 polypeptide. IL-15 derivatives also include a polypeptide that comprises the amino acid sequence of a naturally occurring or wild type mature form of a mammalian IL-15 polypeptide and a heterologous signal peptide amino acid sequence. In one embodiment, an IL-15 derivative is a derivative of a native human IL-15 polypeptide. In another embodiment, an IL-15 derivative is a derivative of an immature or precursor form of naturally occurring or wild type human IL-15 polypeptide. In another embodiment, an IL-15 derivative is a derivative of a mature form of naturally occurring or wild type human IL-15 polypeptide. In another embodiment, an IL-15 derivative is the IL-15N72D described in, e.g. Zhu et al., (2009), J. Immunol. 183: 3598 or U.S. Pat. No. 8,163,879. In another embodiment, an IL-15 derivative is one of the IL-15 variants described in U.S. Pat. No. 8,163,879. In one embodiment, an IL-15 derivative is isolated or purified.

Suitably, IL-15 derivatives retain at least 75%, at least 80%, at least 85%, at least 90%, at least 95%, at least 98% or at least 99% of the function of native mammalian IL-15 polypeptide to bind IL-15Rα polypeptide, as measured by assays known in the art, e.g. ELISA, SPR (e.g. BIAcore™), co-immunoprecipitation. Suitably, IL-15 derivatives retain at least 75%, at least 80%, at least 85%, at least 90%, at least 95%, at least 98% or at least 99% of the function of native mammalian IL-15 polypeptide to induce IL-15-mediated signal transduction, as measured by assays known in the art, e.g. electromobility shift assays, ELISAs or other immunoassays. Suitably, IL-15 derivatives bind to IL-15Rα and/or IL-15Rβγ as assessed by, e.g. ligand/receptor binding assays known in the art. Percent identity can be determined using any method known to one of skill in the art and as described supra.

### IL-15Rα

As used herein, the terms “IL-15Rα” and “interleukin-15 receptor alpha” refer to a native IL-15Rα, an IL-15Rα derivative, or a native IL-15Rα and an IL-15Rα derivative. As used herein, the terms “native IL-15Rα” and “native interleukin-15 receptor alpha” in the context of proteins or polypeptides refer to any naturally occurring and wild type mammalian interleukin-15 receptor alpha (“IL-15Rα”) amino acid sequence, including immature or precursor and mature forms and naturally occurring isoforms. Non-limiting examples of GeneBank Accession Nos. for the amino acid sequence of various native mammalian IL-15Rα include NP_002180 (human), ABK41438 (Macaca mulatta), NP_032384 (Mus musculus), Q60819 (Mus musculus), CAI41082 (human). The amino acid sequence of the immature form of the native full length human IL-15Rα, which comprises the signal peptide (underlined) and the mature human native IL-15Rα (italicized), as provided in SEQ ID NO: 3 in Table 1. The amino acid sequence of the immature form of the native soluble human IL-15Rα, which comprises the signal peptide (underlined) and the mature human native soluble IL-15Rα (italicized), as provided in SEQ ID NO: 4 in Table 1. In some embodiments, native IL-15Rα is the immature form of a naturally occurring or wild type mammalian IL-15Rα polypeptide. In other embodiments, native IL-15Rα is the mature form of a naturally occurring or wild type mammalian IL-15Rα polypeptide. In certain embodiments, native IL-15Rα is the naturally occurring or wild type soluble form of mammalian IL-15Rα polypeptide. In other embodiments, native IL-15Rα is the full-length form of a naturally occurring or wild type mammalian IL-15Rα polypeptide. In a specific embodiment, native IL-15Rα is the immature form of a naturally occurring or wild type human IL-15Rα polypeptide. In another embodiment, native IL-15Rα is the mature form of a naturally occurring or wild type human IL-15Rα polypeptide. In certain embodiments, native IL-15Rα is the naturally occurring or wild type soluble form of human IL-15Rα polypeptide. In other embodiments, native IL-15Rα is the full-length form of a naturally occurring or wild type human IL-15Rα polypeptide. In one embodiment, a native IL-15Rα protein or polypeptide is isolated or purified.

In a particular embodiment, the soluble form of human IL-15Rα comprises an amino acid sequence of

(SEQ ID NO: 5)
ITCPPPMSVEHADIWVKSYSLYSRERYICNSGFKRKAGTSSLTECVLNKA
TNVAHWTTPSLKCIRDPALVHQRPAPPSTVTTAGVTPQPESLSPSGKEPA
ASSPSSNNTAATTAAIVPGSQLMPSKSPSTGTTEISSHESSHGTPSQTTA
KNWELTASASHQPPGVYPQG.

As used herein, the terms “IL-15Rα derivative” and “interleukin-15 receptor alpha derivative” in the context of a protein or polypeptide refer to: (a) a polypeptide that is at least 75%, at least 80%, at least 85%, at least 90%, at least 95%, at least 98% or at least 99% identical to a native mammalian IL-15 polypeptide; (b) a polypeptide that contains 1, 2, 3, 4, 5, 6, 7, 8, 9, 10, 11, 12, 13, 14, 15, 16, 17, 18, 19, 20 or more amino acid mutations (i.e. additions, deletions and/or substitutions) relative to a native mammalian IL-15Rα polypeptide; (c) a fragment of a native mammalian IL-15Rα polypeptide; and/or (d) a specific IL-15Rα derivative described herein. IL-15Rα derivatives also include a polypeptide that comprises the amino acid sequence of a naturally occurring or wild type mature form of mammalian IL-15Rα polypeptide and a heterologous signal peptide amino acid sequence. In one embodiment, an IL-15Rα derivative is a derivative of a native human IL-15Rα polypeptide. In one embodiment, an IL-15Rα derivative is a derivative of an immature form of naturally occurring or wild type human IL-15 polypeptide. In one embodiment, an IL-15Rα derivative is a derivative of a mature form of naturally occurring or wild type human IL-15 polypeptide. In one embodiment, an IL-15Rα derivative is a soluble form of a native mammalian IL-15Rα polypeptide. In other words, in certain embodiments, an IL-15Rα derivative includes soluble forms of native mammalian IL-15Rα, wherein those soluble forms are not naturally occurring. Other examples of IL-15Rα derivatives include the truncated, soluble forms of native human IL-15Rα. In one embodiment, an IL-15Rα derivative is purified or isolated.

Suitably, IL-15Rα derivatives retain at least 75%, at least 80%, at least 85%, at least 90%, at least 95%, at least 98% or at least 99% of the function of a native mammalian IL-15Rα polypeptide to bind an IL-15 polypeptide, as measured by assays known in the art, e.g. ELISA, SPR (BIAcore™), co-immunoprecipitation. In one embodiment, IL-15Rα derivatives retain at least 75%, at least 80%, at least 85%, at least 90%, at least 95%, at least 98% or at least 99% of the function of a native mammalian IL-15Rα polypeptide to induce IL-15-mediated signal transduction, as measured by assays known in the art, e.g. electromobility shift assays, ELISAs and other immunoassays. In one embodiment, IL-15Rα derivatives bind to IL-15 as assessed by methods known in the art, such as, ELISAs.

Provided herein is the naturally occurring or wild type soluble form of human IL-15Rα. Also provided herein are specific IL-15Rα derivatives that are truncated, soluble forms of human IL-15Rα. These specific IL-15Rα derivatives and the naturally occurring or wild type soluble form of human IL-15Rα are based, in part, on the identification of the proteolytic cleavage site of human IL-15Rα. Further provided herein are soluble forms of IL-15Rα that are characterized based upon glycosylation of the IL-15Rα.

The proteolytic cleavage of human IL-15Rα takes place between Glyl70 and His171 which are in shown in bold and underlined in the provided amino acid sequence of the immature form of the native full length human IL-15Rα: MAPRRARGCR TLGLPALLLL LLLRPPATRG ITCPPPMSVE HADIWVKSYS LYSRERYICN SGFKRKAGTS SLTECVLNKA TNVAHWTTPS LKCIRDPALV HQRPAPPSTV TTAGVTPQPE SLSPSGKEPA ASSPSSNNTAATTAAIVPGS QLMPSKSPST GTTEISSHES SHGTPSQTTA KNWELTASAS HQPPGVYPQGHSDTTVAIST STVLLCGLSA VSLLACYLKS RQTPPLASVE MEAMEALPVT WGTSSRDEDL ENCSHHL (SEQ ID NO: 3 in Table 1).

Accordingly, provided herein is a soluble form of human IL-15Rα (e.g. a purified soluble form of human IL-15Rα), wherein the amino acid sequence of the soluble form of human IL-15Rα terminates at the site of the proteolytic cleavage of the native membrane-bound human IL-15Rα. In one embodiment, provided herein is a soluble form of human IL-15Rα (e.g. a purified soluble form of human IL-15Rα), wherein the amino acid sequence of the soluble form of human IL-15Rα terminates with PQG (SEQ ID NO: 11 in Table 1), wherein G is Gly170. In one embodiment, provided herein is a soluble form of human IL-15Rα (e.g. a purified soluble form of human IL-15Rα) which has the amino acid sequence shown in SEQ ID NO: 4 in Table 1. In one embodiment, provided herein is an IL-15Rα derivative (e.g. a purified and/or soluble form of IL-15Rα derivative), which is a polypeptide that: (i) is at least 75%, at least 80%, at least 85%, at least 90%, at least 95%, at least 98% or at least 99% identical to SEQ ID NO: 4 in Table 1; and (ii) terminates with the amino acid sequence PQG (SEQ ID NO: 11 in Table 1). In one embodiment, provided herein is a soluble form of human IL-15Rα (e.g. a purified soluble form of human IL-15Rα) which has the amino acid sequence of SEQ ID NO: 5 in Table 1). In some embodiments, provided herein is an IL-15Rα derivative (e.g. a purified and/or soluble form of an IL-15Rα derivative), which is a polypeptide that is at least 75%, at least 80%, at least 85%, at least 90%, at least 95%, at least 98% or at least 99% identical to SEQ ID NO: 5 in Table 1, and, optionally, wherein the amino acid sequence of the soluble form of the IL-15Rα derivative terminates with PQG (SEQ ID NO: 11 in Table 1).

In one embodiment, provided herein is an IL-15Rα derivative of naturally occurring or wild type human IL-15Rα, wherein the IL-15Rα derivative is soluble and: (a) the last amino acids at the C-terminal end of the IL-15Rα derivative consist of amino acid residues PQGHSDTT (SEQ ID NO: 6 in Table 1); (b) the last amino acids at the C-terminal end of the IL-15Rα derivative consist of amino acid residues PQGHSDT (SEQ ID NO: 7 in Table 1); (c) the last amino acids at the C-terminal end of the IL-15Rα derivative consist of amino acid residues PQGHSD (SEQ ID NO: 8 in Table 1); (d) the last amino acids at the C-terminal end of the IL-15Rα derivative consist of amino acid residues PQGHS (SEQ ID NO: 9 in Table 1); or (e) the last amino acids at the C-terminal end of the IL-15Rα derivative consist of amino acid residues PQGH (SEQ ID NO: 10 in Table 1). In one embodiment, the amino acid sequences of these IL-15Rα derivatives are at least 75%, at least 85%, at least 90%, at least 95%, at least 96%, at least 97%, at least 98%, or at least 99% identical to the amino acid sequence of SEQ ID NO: 12 in Table 1. In one embodiment, these IL-15Rα derivatives are purified.

Also provided herein are glycosylated forms of IL-15Rα (e.g. purified glycosylated forms of IL-15Rα), wherein the glycosylation of the IL-15Rα accounts for at least 20%, at least 25%, at least 30%, at least 35%, at least 40%, at least 45%, at least 50%, or 20% to 25%, 20% to 30%, 25% to 30%, 25% to 35%, 30% to 35%, 30% to 40%, 35% to 40%, 35% to 45%, 40% to 50%, 45% to 50%, 20% to 40%, or 25% to 50% of the mass (molecular weight) of the IL-15Rα as assessed by techniques known to one of skill in the art. The percentage of the mass (molecular weight) of IL-15Rα (e.g. purified IL-15Rα) that glycosylation of IL-15Rα accounts for can be determined using, for example and without limitation, gel electrophoresis and quantitative densitometry of the gels, and comparison of the average mass (molecular weight) of a glycosylated form of IL-15Rα (e.g. a purified glycosylated form of IL-15Rα) to the non-glycosylated form of IL-15Rα (e.g. a purified non-glycosylated form of IL-15Rα). In one embodiment, the average mass (molecular weight) of IL-15Rα (e.g. purified IL-15Rα) is determined using MALDI-TOF MS spectrum on Voyager De-Pro equipped with CovalX HM-1 high mass detector using sinapic acid as matrix, and the mass of a glycosylated form of IL-15Rα (e.g. purified glycosylated form of IL-15Rα) is compared to the mass of the non-glycosylated form of IL-15Rα (e.g. purified non-glycosylated form of IL-15Rα) to determine the percentage of the mass that glycosylation accounts for.

Also provided herein are glycosylated forms of IL-15Rα, wherein the IL-15Rα is glycosylated (N- or O-glycosylated) at certain amino acid residues. In one embodiment, provided herein is a human IL-15Rα which is glycosylated at one, two, three, four, five, six, seven, or all, of the following glycosylation sites: (i) O-glycosylation on threonine at position 5 of the amino acid sequence NWELTASASHQPPGVYPQG (SEQ ID NO: 13 in Table 1) in the IL-15Rα; (ii) 0-glycosylation on serine at position 7 of the amino acid sequence NWELTASASHQPPGVYPQG (SEQ ID NO: 13 in Table 1) in the IL-15Rα; (iii) N-glycosylation on serine at position 8 of the amino acid sequence ITCPPPMSVEHADIWVK (SEQ ID NO: 14 in Table 1) in the IL-15Rα, or serine at position 8 of the amino acid sequence ITCPPPMSVEHADIWVKSYSLYSRERYICNS (SEQ ID NO: 15 in Table 1) in the IL-15Rα; (iv) N-glycosylation on Ser 18 of amino acid sequence ITCPPPMSVEHADIWVKSYSLYSRERYICNS (SEQ ID NO: 15 in Table 1) in the IL-15Rα; (v) N-glycosylation on serine at position 20 of the amino acid sequence ITCPPPMSVEHADIWVKSYSLYSRERYICNS (SEQ ID NO: 15 in Table 1) in the IL-15Rα; (vi) N-glycosylation on serine at position 23 of the amino acid sequence ITCPPPMSVEHADIWVKSYSLYSRERYICNS (SEQ ID NO: 15 in Table 1) in the IL-15Rα; and/or (vii) N-glycosylated on serine at position 31 of the amino acid sequence ITCPPPMSVEHADIWVKSYSLYSRERYICNS (SEQ ID NO: 15 in Table 1) in the IL-15Rα. In one embodiment, the glycosylated IL-15Rα is a native human IL-15Rα. In one embodiment, the glycosylated IL-15Rα is an IL-15Rα derivative of naturally occurring or wild type human IL-15Rα. In one embodiment, the glycosylated IL-15Rα is a native soluble human IL-15Rα, such as SEQ ID NO: 4 or 5 in Table 1. In one embodiment, the glycosylated IL-15Rα is an IL-15Rα derivative that is a soluble form of human IL-15Rα. In one embodiment, the glycosylated IL-15Rα is purified or isolated. IL-15/IL-15Rα complex

As used herein, the term “IL-15/IL-15Rα complex”, “IL-15/IL-15Rα heterocomplex” or “hen-15” refers to a complex comprising IL-15 and IL-15Rα covalently or noncovalently bound to each other. In a preferred embodiment, the IL-15Rα has a high affinity for IL-15, e.g. KD of 10 to 50 pM as measured by a technique known in the art, e.g. KinExA assay, surface plasma resonance (e.g. BIAcore™ assay). In one embodiment, the IL-15/IL-15Rα complex induces IL-15-mediated signal transduction, as measured by assays well-known in the art, e.g. electromobility shift assays, ELISAs and other immunoassays. In one embodiment, the IL-15/IL-15Rα complex retains the ability to specifically bind to the βγ chain. In one embodiment, the IL-15/IL-15Rα complex is isolated from a cell.

Provided herein are complexes that bind to the βγ subunits of the IL-15 receptor, induce IL-15 signal transduction (e.g. Jak/Stat signal transduction) and enhance IL-15-mediated immune function, wherein the complexes comprise IL-15 covalently or noncovalently bound to interleukin-15 receptor alpha (“IL-15Rα”), also referred herein as a “IL-15/IL-15Rα complex” or “IL-15/IL-15Rα heterocomplex”. The IL-15/IL-15Rα complex is able to bind to the βγ receptor complex.

The IL-15/IL-15Rα complexes can be composed of native IL-15 or an IL-15 derivative and native IL-15Rα or an IL-15Rα derivative. In one embodiment, the IL-15/IL-15Rα complex comprises native IL-15 or an IL-15 derivative and an IL-15Rα described above.

In one embodiment, the IL-15/IL-15Rα complex comprises human IL-15 complexed with a soluble form of human IL-15 Rα. The complex can comprise IL-15 covalently or noncovalently bound to a soluble form of IL-15 Rα. In a preferred embodiment, the human IL-15 is noncovalently bound to a soluble form of IL-15 Rα. In a particularly preferred embodiment, the IL-15/IL-15Rα complex comprises human IL-15 comprising SEQ ID NO: 2 non-covalently bound to the soluble form of human IL-15 Rα comprising SEQ ID NO: 5.

In one embodiment, the IL-15/IL-15Rα complex comprises native IL-15 or an IL-15Rα derivative and native soluble IL-15Rα (e.g. native soluble human IL-15Rα). In one embodiment, the IL-15/IL-15Rα complex is composed of an IL-15 derivative and an IL-15Rα derivative. In one embodiment, the IL-15/IL-15Rα complex is composed of native IL-15 and an IL-15Rα derivative. In one embodiment, the IL-15Rα derivative is a soluble form of IL-15Rα. Specific examples of soluble forms of IL-15Rα are described above. In one embodiment, the soluble form of IL-15Rα lacks the transmembrane domain of native IL-15Rα, and optionally, the intracellular domain of native IL-15Rα. In one embodiment, the IL-15Rα derivative is the extracellular domain of native IL-15Rα or a fragment thereof. In one embodiment, the IL-15Rα derivative is a fragment of the extracellular domain comprising the sushi domain or exon 2 of native IL-15Rα. In one embodiment, the IL-15Rα derivative comprises a fragment of the extracellular domain comprising the sushi domain or exon 2 of native IL-15Rα and at least one amino acid that is encoded by exon 3. In one embodiment, the IL-15Rα derivative comprises a fragment of the extracellular domain comprising the sushi domain or exon 2 of native IL-15Rα and an IL-15Rα hinge region or a fragment thereof. In one embodiment, the IL-15Rα comprises the amino acid sequence of SEQ ID NO: 5 in Table 1.

In one embodiment, the IL-15Rα derivative comprises a mutation in the extracellular domain cleavage site that inhibits cleavage by an endogenous protease that cleaves native IL-15Rα. In one embodiment, the extracellular domain cleavage site of IL-15Rα is replaced with a cleavage site that is recognized and cleaved by a heterologous known protease.

In one embodiment, the IL-15 is encoded by a nucleic acid sequence optimized to enhance expression of IL-15, e.g. using methods as described in WO 2007/084342 and WO 2010/020047; and U.S. Pat. Nos. 5,965,726; 6,174,666; 6,291,664; 6,414,132; and 6,794,498.

In one embodiment, provided herein is an IL-15/IL-15Rα complex comprising human IL-15Rα which is glycosylated at one, two, three, four, five, six, seven, or all, of the glycosylation sites as described supra and with reference to SEQ ID NOs: 13, 14 and 15 in Table 1. In one embodiment, the glycosylated IL-15Rα is a native human IL-15Rα. In one embodiment, the glycosylated IL-15Rα is an IL-15Rα derivative of naturally occurring or wild type human IL-15Rα. In one embodiment, the glycosylated IL-15Rα is a native soluble human IL-15Rα, such as SEQ ID NO: 4 or 5 in Table 1. In one embodiment, the glycosylated IL-15Rα is an IL-15Rα derivative that is a soluble form of human IL-15Rα. In one embodiment, the IL-15/IL-15Rα complex is purified or isolated.

In addition to IL-15 and IL-15Rα, the IL-15/IL-15Rα complexes may comprise a heterologous molecule. In some embodiments, the heterologous molecule increases protein stability. Non-limiting examples of such molecules include polyethylene glycol (PEG), Fc domain of an IgG immunoglobulin or a fragment thereof, or albumin that increase the half-life of IL-15 or IL-15Rα in vivo. In some embodiments, IL-15Rα is conjugated/fused to the Fc domain of an immunoglobulin (e.g. an IgG1) or a fragment thereof. In a specific embodiment, the IL-15RαFc fusion protein comprises the amino acid sequence of SEQ ID NO: 16 or 17 in Table 1. In another embodiment, the IL-15RαFc fusion protein is the IL-15Rα/Fc fusion protein described in Han et al., (2011), Cytokine 56: 804-810, U.S. Pat. No. 8,507,222 or U.S. Pat. No. 8,124,084. In those IL-15/IL-15Rα complexes comprising a heterologous molecule, the heterologous molecule can be conjugated to IL-15 and/or IL-15Rα. In one embodiment, the heterologous molecule is conjugated to IL-15Rα. In another embodiment, the heterologous molecule is conjugated to IL-15. In another embodiment, the heterologous molecule is conjugated to IL-15Rα and conjugated to IL-15.

TABLE 1
Sequence Table

| SEQ | | |
|---|---|---|
| ID | | |
| NO | Description | Sequence |
| 1 | Human IL- | MRISKPHLRSISIQCYLCLLLNSHFLTEAGIHVFILGCFSAGLPKTEANW |
| | 15 (with | VN |
| | signal | VISDLKKIEDLIQSMHIDATLYTESDVHPSCKVTAMKCFLLELQVISLES |
| | peptide | GD |
| | underlined) | ASIHDTVENLIILANNSLSSNGNVTESGCKECEELEEKNIKEFLQSFVHI |
| | | VQ |
| | | MFINTS |
| 2 | Mature | NWVNVISDLKKIEDLIQSMHIDATLYTESDVHPSCKVTAMKCFLLELQV |
| | human | ISLESGD |
| | native IL-15 | IASIHDTVENLIILANNSLSSNGNVTESGCKECEELEEKNIKEFLQSFVHI |
| | | VQMFINTS |
| 3 | Human IL- | MAPRRARGCRTLGLPALLLLLLLRPPATRGITCPPPMSVEHADIWVKS |
| | 15Rα (with | YSLYSR |
| | signal | ERYICNSGFKRKAGTSSLTECVLNKATNVAHWTTPSLKCIRDPALVHQ |
| | peptide | RPAPPS |
| | underlined) | TVTTAGVTPQPESLSPSGKEPAASSPSSNNTAATTAAIVPGSQLMPS |
| | | KSPSTGT |
| | | TEISSHESSHGTPSQTTAKNWELTASASHQPPGVYPQGHSDTTVAIS |
| | | TSTVLLC |
| | | GLSAVSLLACYLKSRQTPPLASVEMEAMEALPVTWGTSSRDEDLENC |
| | | SHHL |
| 4 | Human | MAPRRARGCRTLGLPALLLLLLLRPPATRGITCPPPMSVEHADIWVKS |
| | soluble IL- | YSLYSR |
| | 15Rα (with | ERYICNSGFKRKAGTSSLTECVLNKATNVAHWTTPSLKCIRDPALVHQ |
| | signal | RPAPPS |
| | peptide | TVTTAGVTPQPESLSPSGKEPAASSPSSNNTAATTAAIVPGSQLMPS |
| | underlined) | KSPSTGT |
| | | TEISSHESSHGTPSQTTAKNWELTASASHQPPGVYPQG |
| 5 | Mature | ITCPPPMSVEHADIWVKSYSLYSRERYICNSGFKRKAGTSSLTECVLN |
| | human | KATNVAHW |
| | soluble IL- | TTPSLKCIRDPALVHQRPAPPSTVTTAGVTPQPESLSPSGKEPAASSP |
| | 15Rα | SSNNTAATTA |
| | | AIVPGSQLMPSKSPSTGTTEISSHESSHGTPSQTTAKNWELTASASH |
| | | QPPGVYPQG |
| 6 | C-terminal | PQGHSDTT |
| | of soluble | |
| | human IL- | |
| | 15Rα | |
| 7 | C-terminal | PQGHSDT |
| | of soluble | |
| | human IL- | |
| | 15Rα | |
| 8 | C-terminal | PQGHSD |
| | of soluble | |
| | human IL- | |
| | 15Rα | |
| 9 | C-terminal | PQGHS |
| | of soluble | |
| | human IL- | |
| | 15Rα | |
| 10 | C-terminal | PQGH |
| | of soluble | |
| | human IL- | |
| | 15Rα | |
| 11 | C-terminal | PQG |
| | of soluble | |
| | human IL- | |
| | 15Rα | |
| 12 | Human | ITCPPPMSVEHADIWVKSYSLYSRERYICNSGFKRKAGTSSLTECVLN |
| | soluble IL- | KATN |
| | 15Rα | VAHWTTPSLKCIRDPALVHQRPAPPSTVTTAGVTPQPESLSPSGKEP |
| | | AASSP |
| | | SSNNTAATTAAIVPGSQLMPSKSPSTGTTEISSHESSHGTPSQTTAKN |
| | | WELT |
| | | ASASHQPPGVYPQGHSDTT |
| 13 | IL-15Rα O- | NWELTASASHQPPGVYPQG |
| | glycosylation | |
| 14 | IL-15Rα N- | ITCPPPMSVEHADIWVK |
| | glycosylation | |
| 15 | IL-15Rα N- | ITCPPPMSVEHADIWVKSYSLYSRERYICNS |
| | glycosylation | |
| 16 | synthetic | MAPRRARGCRTLGLPALLLLLLLRPPATRGITCPPPMSVEHADIWVKS |
| | sIL- | YSLYS |
| | 15Rαlpha- | RERYICNSGFKRKAGTSSLTECVLNKATNVAHWTTPSLKCIRDPALVH |
| | Fc fusion | QRPAP |
| | protein | PSTVTTAGVTPQPESLSPSGKEPAASSPSSNNTAATTAAIVPGSQLM |
| | hulL15sRa205- | PSKSPS |
| | Fc | TGTTEISSHESSHGTPSQTTAKNWELTASASHQPPGVYPQGHSDTTP |
| | | KSCDKT |
| | | HTCPPCPAPELLGGPSVFLFPPKPKDTLMISRTPEVTCVVVDVSHEDP |
| | | EVKFN |
| | | WYVDGVEVHNAKTKPREEQYNSTYRVVSVLTVLHQDWLNGKEYKCK |
| | | VSNKALP |
| | | APIEKTISKAKGQPREPQVYTLPPSRDELTKNQVSLTCLVKGFYPSDIA |
| | | VEWE |
| | | SNGQPENNYKTTPPVLDSDGSFFLYSKLTVDKSRWQQGNVFSCSVM |
| | | HEALHNH |
| | | YTQKSLSLSPGK |
| 17 | synthetic | MAPRRARGCRTLGLPALLLLLLLRPPATRGITCPPPMSVEHADIWVKS |
| | sIL- | YSLYS |
| | 15Rαlpha- | RERYICNSGFKRKAGTSSLTECVLNKATNVAHWTTPSLKCIRDPALVH |
| | Fc fusion | QRPAP |
| | protein | PSTVTTAGVTPQPESLSPSGKEPAASSPSSNNTAATTAAIVPGSQLM |
| | hulL15sRa200- | PSKSPS |
| | Fc | TGTTEISSHESSHGTPSQTTAKNWELTASASHQPPGVYPQGPKSCD |
| | | KTHTCPP |
| | | VCPAPELLGGPSVFLFPPKPKDTLMISRTPEVTCVVVDVSHEDPEVKFN |
| | | WYVDG |
| | | VEVHNAKTKPREEQYNSTYRVVSVLTVLHQDWLNGKEYKCKVSNKA |
| | | LPAPIEK |
| | | TISKAKGQPREPQVYTLPPSRDELTKNQVSLTCLVKGFYPSDIAVEWE |
| | | SNGQP |
| | | ENNYKTTPPVLDSDGSFFLYSKLTVDKSRWQQGNVFSCSVMHEALH |
| | | NHYTQKS |
| | | LSLSPGK |

The components of an IL-15/IL-15Rα complex can be directly fused, using either non-covalent bonds or covalent bonds (e.g. by combining amino acid sequences via peptide bonds), and/or can be combined using one or more linkers. Linkers suitable for preparing the IL-15/IL-15Rα complexes comprise peptides, alkyl groups, chemically substituted alkyl groups, polymers, or any other covalently-bonded or non-covalently bonded chemical substance capable of binding together two or more components. Polymer linkers comprise any polymers known in the art, including polyethylene glycol (PEG). In some embodiments, the linker is a peptide that is 1, 2, 3, 4, 5, 6, 7, 8, 9, 10, 11, 12, 13, 14, 15, 16, 17, 18, 19, 20 or more amino acids long. In one embodiment, the linker is long enough to preserve the ability of IL-15 to bind to the IL-15Rα. In other embodiments, the linker is long enough to preserve the ability of the IL-15/IL-15Rα complex to bind to the βγ receptor complex and to act as an agonist to mediate IL-15 signal transduction.

In some embodiments, IL-15/IL-15Rα complexes are pre-coupled prior to use in the methods described herein (e.g. prior to contacting cells with the IL-15/IL-15Rα complexes or prior to administering the IL-15/IL-15Rα complexes to a subject). In other embodiments, the IL-15/IL-15Rα complexes are not pre-coupled prior to use in the methods described herein.

In a specific embodiment, the IL-15/IL-15Rα complex enhances or induces immune function in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the immune function in a subject not administered the IL-15/IL-15Rα complex using assays known in the art, e.g. ELISPOT, ELISA, and cell proliferation assays. In a specific embodiment, the immune function is cytokine release (e.g. interferon-gamma, IL-2, IL-5, IL-10, IL-12, or transforming growth factor (TGF)-beta). In some embodiments, the IL-15 mediated immune function is NK cell proliferation, which can be assayed, e.g. by flow cytometry to detect the number of cells expressing markers of NK cells (e.g. CD56). In some embodiments, the IL-15 mediated immune function is antibody production, which can be assayed, e.g. by ELISA. In some embodiments, the IL-15 mediated immune function is effector function, which can be assayed, e.g. by a cytotoxicity assay or other assays known in the art.

In specific embodiments, examples of immune function enhanced by the IL-15/IL-15Rα complex include the proliferation/expansion of lymphocytes (e.g. increase in the number of lymphocytes), inhibition of apoptosis of lymphocytes, activation of dendritic cells (or antigen presenting cells), and/or antigen presentation. In particular embodiments, immune function enhanced by the IL-15/IL-15Rα complex is proliferation/expansion in the number of or activation of CD4+ T cells (e.g. Th1 and Th2 helper T cells), CD8+ T cells (e.g. cytotoxic T lymphocytes, alpha/beta T cells, and gamma/delta T cells), B cells (e.g. plasma cells), memory T cells, memory B cells, dendritic cells (immature or mature), antigen presenting cells, macrophages, mast cells, natural killer T cells (NKT cells), tumor-resident T cells, CD122+ T cells, or natural killer cells (NK cells). In one embodiment, the IL-15/IL-15Rα complex enhances the proliferation/expansion or number of lymphocyte progenitors. In some embodiments, the IL-15/IL-15Rα complex increases the number of CD4+ T cells (e.g. Th1 and Th2 helper T cells), CD8+ T cells (e.g. cytotoxic T lymphocytes, alpha/beta T cells, and gamma/delta T cells), B cells (e.g. plasma cells), memory T cells, memory B cells, dendritic cells (immature or mature), antigen presenting cells, macrophages, mast cells, natural killer T cells (NKT cells), tumor-resident T cells, CD122+ T cells, or natural killer cells (NK cells) by approximately 1 fold, approximately 2 fold, approximately 3 fold, approximately 4 fold, approximately 5 fold, approximately 6 fold, approximately 7 fold, approximately 8 fold, approximately 9 fold, approximately 10 fold, approximately 20 fold, or more relative a negative control (e.g. number of the respective cells not treated, cultured, or contacted with an IL-15/IL-15Rα complex described herein).

In a specific embodiment, the IL-15/IL-15Rα complex increases the expression of IL-2 on whole blood activated by Staphylococcal enterotoxin B (SEB). For example, the IL-15/IL-15Rα complex increases the expression of IL-2 by at least about 2 fold, about 3 fold, about 4 fold, or about 5 fold, compared to the expression of IL-2 when SEB alone is used.

As used herein, the terms “subject” and “patient” include any human or nonhuman animal. The term “nonhuman animal” includes all vertebrates, e.g. mammals and non-mammals, such as nonhuman primates, sheep, dogs, cats, horses, cows, chickens, amphibians, reptiles, etc. In a preferred embodiment, the subject is a human patient. The terms “subject” and “patient” are used interchangeably herein.

The term “pharmaceutical formulation” or “pharmaceutical composition” refers to a preparation that contains an heterodimeric IL-15/IL-15Rα complex, e.g. as described herein, in such form as to permit the biological activity of the complex to be effective, and which contains no additional components which are unacceptably toxic to a subject to which the formulation would be administered.

The term “pharmaceutically acceptable” as used herein refers to those compounds, materials, compositions and/or dosage forms, which are, within the scope of sound medical judgment, suitable for contact with the tissues of a subject, e.g. a mammal or human, without excessive toxicity, irritation, allergic response and other problems or complications commensurate with a reasonable benefit/risk ratio and which does not interfere with the effectiveness of the biological activity of the active ingredient(s).

The term “administering” in relation to a compound, e.g. IL-15/IL-15Rα complex or another agent, is used to refer to delivery of that compound to a patient by any route.

As used herein, a “therapeutically effective amount” refers to an amount of IL-15/IL-15Rα complex, e.g. as disclosed herein, that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g. IL-15/IL-15Rα complex, e.g. as disclosed herein) administered alone, the term refers to that ingredient alone. When applied to a combination, the term refers to combined amounts of the active ingredients that result in the therapeutic effect, whether administered in combination, serially or simultaneously.

By “a combination” or “in combination with” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

The terms “treat”, “treatment” and “treating” refer to the reduction or amelioration of the progression, severity and/or duration of a disorder, e.g. a proliferative disorder, or the amelioration of one or more symptoms (preferably, one or more discernible symptoms) of the disorder resulting from the administration of one or more therapies. For example, the terms “treat”, “treatment” and “treating” refer to the amelioration of at least one measurable physical parameter of a proliferative disorder, such as growth of a tumor, not necessarily discernible by the patient. Suitably, the terms “treat”, “treatment” and “treating” refer to the inhibition of the progression of a proliferative disorder, either physically by, e.g. stabilization of a discernible symptom, physiologically by, e.g. stabilization of a physical parameter, or both. Suitably, the terms “treat”, “treatment” and “treating” refer to the reduction or stabilization of tumor size or cancerous cell count.

The terms “disease” and “disorder” are used interchangeably to refer to a condition, in particular, a pathological condition. In certain embodiments, the terms “disease” and “disorder” are used interchangeably to refer to a disease affected by IL-15 signal transduction and/or a disease affected by the promotion of an immune effector response.

As used herein, the terms “therapies” and “therapy” can refer to any protocol(s), method(s), compositions, formulations, and/or agent(s) that can be used in the prevention, treatment, management, or amelioration of a disease, e.g. cancer, infectious disease, lymphopenia, and immunodeficiencies, or a symptom associated therewith. In certain embodiments, the terms “therapies” and “therapy” refer to biological therapy, supportive therapy, and/or other therapies useful in treatment, management, prevention, or amelioration of a disease or a symptom associated therewith known to one of skill in the art.

The term “anti-cancer effect” or “anti-tumor effect” refers to a biological effect which can be manifested by various means, including but not limited to, e.g. a decrease in tumor volume, a decrease in the number of cancer or tumor cells, a decrease in the number of metastases, an increase in life expectancy, decrease in cancer cell or tumor cell proliferation, decrease in cancer cell or tumor cell survival, or amelioration of various physiological symptoms associated with the cancerous condition.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g. both terms encompass solid and liquid, e.g. diffuse or circulating, tumors. As used herein, the term “cancer” or “tumor” includes premalignant, as well as malignant cancers and tumors.

The terms “immune effector” or “effector function” or “response” as used herein, refers to function or response, e.g. of an immune effector cell, that enhances or promotes an immune attack of a target cell. For example, an immune effector function or response refers a property of a T cell or NK cell that promotes killing or the inhibition of growth or proliferation, of a target cell. In the case of a T cell, primary stimulation and co-stimulation are examples of immune effector function or response. Other effector functions of a T cell, for example, are cytolytic activity or helper activity including the secretion of cytokines.

The phrase “means for administering” is used to indicate any available implement for systemically administering a drug to a patient, including, but not limited to, a pre-filled syringe, a vial and syringe, an injection pen, an autoinjector, an intravenous (i.v.) drip and bag, a pump, a patch pump, etc. With such items, a patient may self-administer the drug (i.e., administer the drug on their own behalf) or a physician may administer the drug. In some embodiments of the disclosed methods, kits, and uses, the IL-15/IL-15Rα complex, e.g. as disclosed herein, is delivered to the patient via the i.v. route. In some embodiments of the disclosed methods, kits, and uses, the IL-15/IL-15Rα complex, e.g. as disclosed herein, is delivered to the patient via the subcutaneous (s.c.) route.

When a dose of an IL-15/IL-15Rα complex is referenced herein, the dose is according to the mass of the single-chain IL-15. The single-chain IL-15 equivalent is calculated from (i) the mass of an IL-15/IL-15Rα complex by amino acid analysis and (ii) the ratio of IL-15 to IL-15Rα (e.g. soluble IL-15Rα) in the specific preparation as determined experimentally by RP-HPLC or by amino acid analysis.

The term “pharmaceutical product” means a container (e.g. pen, syringe, bag, pump, etc.) having a pharmaceutical composition disposed within said container. By “container” is meant any means for holding a liquid or solid pharmaceutical composition, e.g. a pen, syringe, vial, autoinjector, patch, etc. to store, transport, and maintain the disclosed compositions. Pharmaceutically acceptable containers for use as part of the disclosed pharmaceutical products include syringes (e.g. available from Beckton Dickinson, Nuova Ompi, et al), stoppered vials, cartridges, autoinjectors, patch pumps and injector pens.

A “stable” composition is one in which the protein or protein complex, e.g. as disclosed herein, essentially retains its stability (e.g. physical stability and/or chemical stability and/or biological activity) upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. A “stable liquid pharmaceutical composition” or a “stable solid pharmaceutical composition” is a pharmaceutical composition with no significant physical, chemical and/or biological changes of the proteins, e.g. of a IL-15/IL-15Rα complex, e.g. as disclosed herein, observed when stored at a refrigerated temperature (about 2° C. to about 8° C.) for at least about 6 months, at least about 12 months, at least about 2 years, or at least about 3 years; or at room temperature (about 20° C. to about 25° C.) for at least about 3 months, for at least about 6 months, and for at least about 1 year; or at stressed conditions (about 40° C.) for at least about 1 month, for at least about 3 months, and for at least about 6 months. Various stability criteria can be used, e.g. no more than 10%, no more than 5%, of protein is degraded (e.g. as measured by SEC purity, RP-HPLC purity, charge heterogeneity by AEX, CE-SDS purity (non-reducing), etc.). Alternatively, stability may be shown if the solution remains clear to slightly opalescent by visual analysis or by using nephelometry. Alternatively, stability can be shown if concentration, pH and osmolality of the composition have no more than +/−10% variation over a given time period, e.g. at least about 3 months, at least about 6 months, and at least about 1 year. Alternatively, stability can be shown using biological assays as described herein. Alternatively, stability may be shown if less than 1%, preferably less than 0.5% aggregates are formed (e.g., as measured by AP-SEC, DP-SEC etc.) over a given time period, e.g., at least 1 month, at least 3 months, at least 6 months, at least 12 months. Alternatively, stability may be shown if, after 6 months storage at 2-8° C., degradation product formation (as measured by RP-HPLC (sum of impurities)) is <about 10%, <about 15%, <about 10% or <about 5%.

A protein, e.g. IL-15 and/or IL-15Rα, e.g. as disclosed herein, or protein complex, e.g. IL-15/IL-15Rα complex, e.g. as disclosed herein, retains its physical stability in a pharmaceutical composition if it shows no significant increase of aggregation, precipitation and/or denaturation, e.g. upon visual examination of color and/or clarity (turbidity), or as measured by UV light scattering, size exclusion chromatography (SEC), SDS-PAGE, dynamic light scattering (DLS) and/or other methods known in the art. In addition, the protein conformation should not be significantly altered, e.g. as evaluated by fluorescence spectroscopy (determines the tertiary structure), circular dichroism spectroscopy (determines the secondary and tertiary structure) and/or by FTIR spectroscopy (determines the secondary structure).

A protein, e.g. IL-15 and/or IL-15Rα, e.g. as disclosed herein, or protein complex, e.g. IL-15/IL-15Rα complex, e.g. as disclosed herein, retains its chemical stability in a pharmaceutical composition if it shows no significant chemical alteration. Chemical stability can be assessed by detecting and/or quantifying chemically altered forms of the protein, e.g. IL-15 and/or IL-15Rα, e.g. as disclosed herein, or protein complex, e.g. IL-15/IL-15Rα complex, e.g. as disclosed herein. Degradation processes that often alter the protein chemical structure include hydrolysis or clipping, e.g. evaluated by methods such as size exclusion chromatography [SEC], SDS-PAGE and/or MALDI-TOF MS, oxidation, e.g. evaluated by methods such as by peptide mapping in conjunction with mass spectroscopy or MALDI-TOF MS, deamidation, e.g. evaluated by methods such as cation-exchange chromatography (CEX, capillary isoelectric focusing, peptide mapping, isoaspartic acid measurement, and isomerization, e.g. evaluated by measuring the isoaspartic acid content, peptide mapping, etc., or other methods known in the art.

A protein, e.g. IL-15 and/or IL-15Rα, e.g. as disclosed herein, or protein complex e.g. IL-15/IL-15Rα complex, e.g. as disclosed herein, retains its biological activity in a pharmaceutical composition, if the biological activity of the protein, e.g. IL-15 and/or IL-15Rα, e.g. as disclosed herein, or protein complex, e.g. IL-15/IL-15Rα complex, e.g. as disclosed herein, at a given time is within a predetermined range of the biological activity exhibited at the time the pharmaceutical composition was prepared. The biological activity of the protein, e.g. IL-15 and/or IL-15Rα, or protein complex, e.g. IL-15/IL-15Rα complex, can be determined, for example, by a cytokine release assay (e.g. interferon-gamma, IL-2, IL-5, IL-10, IL-12, or transforming growth factor (TGF)-beta), NK cell proliferation assay, e.g. as determined by flow cytometry to detect the number of cells expressing markers of NK cells (e.g. CD56), antibody production assay, e.g. which can be determined by ELISA, or effector function assay, e.g. by a cytotoxicity assay. . The biological activity of the protein, e.g. IL-15 and/or IL-15Rα, or protein complex, e.g. IL-15/IL-15Rα complex as described herein, can be determined, for example, by assaying activation of IL-15 receptor on U2OS IL2Rβ/IL2Rγ cells. The activity can be expressed relative to the “original activity” by comparing the activity of a sample comprising protein, e.g. IL-15 and/or IL-15Rα, or protein complex, e.g. IL-15/IL-15Rα complex as described herein, upon storage and comparing said sample with a reference sample.

As used herein, “purity by RP-HPLC” refers to the percentage of IL-15 related peaks and IL-15Rα related peaks in RP-HPLC and can be used to assess the stability of protein, IL-15 and IL-15Rα, e.g. as disclosed herein. RP-HPLC is used to separate protein IL-15 and IL-15Rα, e.g. as disclosed herein and its variants according to their hydrophobicity. Other peaks by RP-HPLC may contain fragmented, isomerized, and oxidized species of IL-15, IL-15Rα and/or of the IL-15/IL-15Rα complex, e.g. as disclosed herein.

As used herein, “charge heterogeneity by AEX” refers to the percentage of basic or acidic variants in AEX and can be used to assess the stability of IL-15, IL-15Rα and/or of the IL-15/IL-15Rα complex, e.g. as disclosed herein. AEX is used to evaluate the charge heterogeneity of the IL-15/IL-15Rα complex, e.g. as disclosed herein, by measuring the percentage of acidic and basic variants.

As used herein, “purity by SEC” refers to the percentage of monomer in SEC and can be used to assess the stability of IL-15, IL-15Rα and/or of the IL-15/IL-15Rα complex, e.g. as disclosed herein. SEC is used to separate monomeric as disclosed herein from aggregates and fragments according to their size under non-denaturing conditions. The sum of peaks eluting prior the main peak are reported as percentage of aggregation products (AP-SEC), the sum of peaks eluting after the main peak as percentage of degradation products (DP-SEC).

As used herein, “purity by CE-SDS” refers to the percentage of intact IL-15Rα, intact IL-15, IL-15 high molecular weight (HMW) species and aglycosylated IL-15 in CE-SDS and can be used to assess the stability of the IL-15/IL-15Rα complex, e.g. as disclosed herein. CE-SDS is used to separate by- and degradation products from IL-15/IL-15Rα complex, e.g. as disclosed herein according to their molecular size under non-reducing conditions. The sum of peaks separated from the identified IL-15Rα and IL-15 related peaks described above is reported as percentage of impurities.

The phrase “liquid pharmaceutical composition” as used herein refers to an aqueous composition that is not reconstituted from a lyophilizate and that contains at least the IL-15/IL-15Rα complex, e.g. as disclosed herein, and at least one additional excipient (e.g. surfactant or buffer). The liquid pharmaceutical composition may include additional excipients (e.g. stabilizer(s)) and additional active ingredient(s). This type of formulation is also referred to as a “ready-to-use” formulation.

As used herein, the term “Iyophilizate” refers to dried (e.g. freeze dried) pharmaceutical compositions largely devoid of water. Techniques for lyophilization of proteins are known in the art, e.g. see Rey & May (2004) Freeze-Drying/Lyophilization of Pharmaceutical & Biological Products ISBN 0824748689. Lyophilizates are reconstituted to give aqueous compositions—usually for immediate use (e.g. within 1-10 days)—as reconstituted lyophilizates tend to have a limited shelf lives.

The phrase “solid pharmaceutical composition” as used herein refers to a pharmaceutical composition that is reconstituted from a lyophilizate prior to administration, and that contains at least the IL-15/IL-15Rα complex, e.g. as disclosed herein, and at least one buffer, at least one stabilizer and at least one tonicity modifier. The solid pharmaceutical composition may include additional excipient(s) and additional active ingredient(s).

The term “buffering agent” us used herein refers to a pharmaceutically acceptable excipient, which stabilizes the pH of a pharmaceutical composition. The buffering agent may be present in a liquid or solid (e.g. lyophilized) formulation of the invention. Suitable buffering agents for use with the disclosed pharmaceutical compositions include, but are not limited to, gluconate buffer, histidine buffer, citrate buffer, phosphate [e.g. sodium and/or potassium] buffer, succinate [e.g. sodium] buffer, acetate buffer [e.g. sodium or potassium], Tris buffer, glycine, arginine and combinations thereof. Buffers are generally used at a concentration of about 1 mM to about 100 mM, of about 10 mM to about 50 mM, of about 15 mM to about 30 mM, of about 20 mM to about 30 mM. Regardless of the buffer used, the pH can be adjusted to a required value, e.g. in the range from about 4.5 to about 8.5, with an acid or a base known in the art, e.g. hydrochloric acid, acetic acid, phosphoric acid, sulfuric acid and citric acid, sodium hydroxide and potassium hydroxide.

Stabilizers assist in preventing oxidation and aggregation of proteins in pharmaceutical compositions. Various analytical methods may be used to assess the stability of a given composition, e.g. RP-HPLC may be used to assay the level of oxidation products (pre-main peaks) in the liquid and/or solid pharmaceutical compositions disclosed herein, while SEC may be used to assay the level of aggregation in the liquid and/or solid pharmaceutical compositions disclosed herein. Suitable stabilizers for use in the disclosed liquid and/or solid pharmaceutical compositions include ionic and non-ionic stabilizers and stabilizers include but are not limited to saccharides (e.g. monosaccharides, disaccharides, trisaccharides and oligosaccharides), amino acids (e.g. glycine, arginine), sugar alcohol/polyols (e.g. mannitol, sorbitol, xylitol, dextran, glycerol, arabitol, propylene glycol, polyethylene glycol), cyclodextrines (e.g. hydroxypropyl-β-cyclodextrine, sulfobutylethyl-β-cyclodextrine, β-cyclodextrine), polyethylene glycols (e.g. PEG 3000, PEG 3350, PEG 4000, PEG 6000), albumins (e.g. human serum albumin (HSA), bovine serum albumin (BSA)), salts (e.g. sodium chloride, magnesium chloride, calcium chloride), chelators (e.g. EDTA), antioxidants (e.g. sodium ascorbate, cysteine, sodium bisulfate, sodium citrate, methionine, benzyl alcohol). More than one stabilizer, selected from the same or from different groups, may be present in the liquid and/or solid pharmaceutical composition.

The term “bulking agent” includes agents that can provide additional structure to a freeze-dried product (e.g. to provide a pharmaceutically acceptable cake). Commonly used bulking agents include mannitol, glycine, lactose, sucrose, and the like. In addition to providing a pharmaceutically acceptable cake, bulking agents also typically impart useful qualities to the solid composition such as modifying the collapse temperature, providing freeze-thaw protection, further enhancing the protein stability over long-term storage, and the like. These agents can also serve as tonicity modifiers and/or stabilizers.

The term “cryoprotectants” generally includes agents that stabilize the protein or protein derivative against freezing-induced stresses. They also typically offer protection during primary and secondary drying, and long-term product storage. Examples of such cryoprotectants are polymers such as dextran and polyethylene glycol; sugars such as sucrose, glucose, trehalose, and lactose; surfactants such as polysorbates; and amino acids such as glycine, arginine, serine, and the like.

The term “lyphoprotectant” includes agents that provide stability to a protein during a drying or ‘dehydration’ process (primary and secondary drying cycles), presumably by providing an amorphous glassy matrix and by binding with the protein or protein complex, e.g. as disclosed herein, through hydrogen bonding, e.g. by replacing the water molecules that are removed during the drying process. This helps to maintain protein conformation, minimize protein degradation during a lyophilization cycle, and improve the long-term stability of the protein or protein derivative. Examples include polyols or sugars such as sucrose and trehalose.

“Reconstitution time” is the time that is required to rehydrate a solid formulation with a liquid, e.g. to provide a particle-free clarified solution.

The term “isotonic” means that the formulation of interest has essentially the same osmolality as human blood. Isotonic formulations generally have an osmolality of about 270-328 mOsm. Slightly hypotonic osmolality in pressure is about250-269 mOsm and slightly hypertonic is about 328-350 mOsm. Osmolality is measured, for example, using a vapor pressure or ice-freezing type osmometer.

Tonicity modifiers useful in the formulations of the present invention include, for example, salts, e.g. NaCl, KCl, MgCl₂, CaCl₂, and the like, and are used to control osmolality. In addition, cryprotecants/lyoprotectants and/or bulking agents such as sucrose, mannitol, glycine, and others can serve as tonicity modifiers.

### Pharmaceutical Compositions

The present disclosure is directed to stable liquid pharmaceutical compositions and solid pharmaceutical compositions comprising at least one IL-15/IL-15Rα complex, e.g. as disclosed herein, which are described supra, and at least one additional excipient, e.g. buffer, surfactant, and stabilizer(s), etc. In some embodiments, the pharmaceutical composition comprises at least two additional excipients, e.g. a buffer and a stabilizer. In some embodiments, the pharmaceutical composition comprises a buffer, at least one stabilizer, and a surfactant.

It is an object of the present invention to provide pharmaceutical compositions comprising at least one IL-15/IL-15Rα complex, e.g. as disclosed herein, which is stable upon storage and delivery. A stable composition is a composition wherein the at least one IL-15/IL-15Rα complex, e.g. as disclosed herein, retains its physical and/or chemical stability and/or retains biological activity upon storage. For example, the pharmaceutical composition should exhibit a shelf life following lyophilization and storage or storage in case of liquid formulation of more than about 6 months, more than about 12 months, more than about 18 months, more than about 24 months, more than about 36 months. The stability of the pharmaceutical composition can be measured using biological activity assays.

In general, a pharmaceutical composition is formulated with excipients that are compatible with the intended route of administration (e.g. oral compositions generally include an inert diluent or an edible carrier). Examples of routes of administration include parenteral (e.g. intravenous), intradermal, subcutaneous, oral (e.g. by mouth or inhalation), transdermal (topical), transmucosal, and rectal. The compositions of this disclosure are suitable for parenteral administration such as intravenous, intramuscular, intraperitoneal, or subcutaneous injection; particularly suitable for subcutaneous injection.

The viscosity of a pharmaceutical composition comprising at least one IL-15/IL-15Rα complex, e.g. as disclosed herein, can be controlled for subcutaneous or intravenous administration. The viscosity can be affected by protein concentration and pH. For example, as the protein concentration increases, the viscosity can increase. An increase in pH can decrease the viscosity of the IL-15/IL-15Rα complex composition. In some compositions, sodium chloride is added to reduce the viscosity of the formulation. Additional components that can affect viscosity of an IL-15/IL-15Rα complex composition are amino acids such as histidine and arginine.

The pharmaceutical composition can be a liquid or a solid. Liquid formulations are aqueous solutions or suspensions, prepared in a suitable aqueous solvent, such as water or an aqueous/organic mixture, such as water alcohol mixtures. Liquid formulations can be kept at room temperature, refrigerated (e.g. 2-8° C.), or frozen (e.g. −20° C. or −70° C.) for storage.

A solid formulation can be prepared in any suitable way and can be in the form of a cake or powder, for example, with the addition of a lyoprotectant. In one aspect, the solid formulation is prepared by drying a liquid formulation as described herein, for example by lyophilization or spray drying. When the formulation is a solid formulation, the formulation can have a moisture content of no more than about 5%, no more than about 4.5%, no more than about 4%, no more than about 3.5%, no more than about 3%, no more than about 2.5%, no more than about 2%, no more than about 1.5%, no more than about 1%, or is substantially anhydrous. A solid formulation can be dissolved, i.e. reconstituted, in a suitable medium or solvent to become liquid suitable for administration. Suitable solvents for reconstituting the solid formulation include water, isotonic saline, buffer, e.g. phosphate-buffered saline, Ringer's (lactated or dextrose) solution, minimal essential medium, alcohol/aqueous solutions, dextrose solution, etc. The amount of solvent can result in a therapeutic protein concentration higher, the same, or lower than the concentration prior to drying.

In some embodiments, the pharmaceutical composition disclosed herein maintains at least about 75%, about 80%, about 85%, about 90% or about 95% purity by RP-HPLC upon storage at about 2° C. to about 8° C. for at least about 6 months, at least about 12 months or at least about 24 months; at least about 75%, at least about 80%, at least about 85%, at least about 90% purity by RP-HPLC upon storage at about 25° C. for at least about 6 months or at least about 12 months; and/or at least about 75%, at least about 80%, at least about 85%, at least about 90%, at least about 95% purity by RP-HPLC upon storage at about 40° C. for at least about 6 months. In some embodiments, a pharmaceutical composition of the disclosure maintains at least about 85% purity by RP-HPLC upon storage at about 2 to about 8° C. for at least about 1 month, at least about 2 months, at least about 3 months, at least about 6 months, at least about 12 months or at least about 24 months. In some embodiments, a pharmaceutical composition of the disclosure maintains at least about 90% purity by RP-HPLC upon storage at about 2° C. to about 8° C. for at least about 1 month, at least about 2 months, at least about 3 months, at least about 6 months, at least about 12 months or at least about 24 months. In some embodiments, a pharmaceutical composition of the disclosure maintains about 95% purity by RP-HPLC upon storage at about 2° C. to about 8° C. for at least about 1 month, at least about 2 months, at least about 3 months, at least about 6 months, at least about 12 months or at least about 24 months. In some embodiments, a pharmaceutical composition of the disclosure maintains about 85% purity by RP-HPLC upon storage at about 25° C. for at least about 1 month, at least about 2 months, at least about 3 months, at least about 6 months, at least about 12 months or at least about 24 months. In some embodiments, a pharmaceutical composition of the disclosure maintains about 90% purity by RP-HPLC upon storage at about 25° C. for at least about 1 month, at least about 2 months, at least about 3 months, at least about 6 months, at least about 12 months or at least about 24 months. In some embodiments, a pharmaceutical composition of the disclosure maintains about 95% purity by RP-HPLC upon storage at about 25° C. for at least about 1 month, at least about 2 months, at least about 3 months, at least about 6 months, at least about 12 months or at least about 24 months. In some embodiments, a pharmaceutical composition of the disclosure maintains about 85% purity by RP-HPLC upon storage at about 40° C. for at least about 1 month, at least about 2 months, at least about 3 months, at least about 6 months, at least about 12 months or at least about 24 months. In some embodiments, a pharmaceutical composition of the disclosure maintains about 90% purity by RP-HPLC upon storage at about 40° C. for at least about 1 month, at least about 2 months, at least about 3 months, at least about 6 months, at least about 12 months or at least about 24 months. In some embodiments, a pharmaceutical composition of the disclosure maintains about 95% purity by RP-HPLC upon storage at about 40° C. for at least about 1 month, at least about 2 months, at least about 3 months, at least about 6 months, at least about 12 months or at least about 24 months.

In some embodiments, the pharmaceutical composition disclosed herein maintains about 25% basic variants and about 75% acidic variants to about 75% basic variant and about 25% acidic variants as assessed by AEX upon storage at about 2° C. to about 8° C. for at least about 6 months, at least about 12 months or at least about 24 months; about 25% basic variants and about 75% acidic variants to about 75% basic variant and about 25% acidic variants as assessed by AEX upon storage at about 20° C. to about 25° C. for at least about 6 months or at least about 12 months; and/or about 25% basic variants and about 75% acidic variants to about 75% basic variant and about 25% acidic variants as assessed by AEX upon storage at about 40° C. for at least about 6 months.

In some embodiments, a pharmaceutical composition of the disclosure comprises less than about 200 particles of >2 μm by PAMAS upon storage at about 2° C. to about 8° C. for at least about 1 month, at least about 2 months, at least about 3 months, at least about 6 months, at least about 12 months or at least about 24 months, and/or less than about 20 particles of >10 μm by PAMAS upon storage at about 2° C. to about 8° C. for at least about 1 month, at least about 2 months, at least about 3 months, at least about 6 months, at least about 12 months or at least about 24 months. In some embodiments, a pharmaceutical composition of the disclosure comprises less than about 200 particles of >2 μm by PAMAS upon storage at about 25° C. for at least about 1 month, at least about 2 months, at least about 3 months, at least about 6 months, at least about 12 months or at least about 24 months, and/or less than about 20 particles of >10 μm by PAMAS upon storage at about 25° C. for at least about 1 month, at least about 2 months, at least about 3 months, at least about 6 months, at least about 12 months or at least about 24 months. In some embodiments, a pharmaceutical composition of the disclosure comprises less than about 200 particles of >2 μm by PAMAS upon storage at about 40° C. for at least about 1 month, at least about 2 months, at least about 3 months, at least about 6 months, at least about 12 months or at least about 24 months, and/or less than about 20 particles of >10 μm by PAMAS upon storage at about 40° C. for at least about 1 month, at least about 2 months, at least about 3 months, at least about 6 months, at least about 12 months or at least about 24 months.

The stability of the pharmaceutical composition can be measured using biological activity assays. Preferably, the biological activity upon storage is about 70% to about 125% of the original activity. Biological activity can be assessed by assaying activation of IL-15 receptor on U2OS IL2Rβ/IL2Rγ cells.

In some embodiments, the liquid pharmaceutical composition disclosed herein maintains biological activity as assessed by activation of IL-15 receptor on U2OS IL2Rβ/IL2Rγ cells upon storage at about 2° C. to about 8° C. for at least about 6 months, at least about 12 months or at least about 24 months, wherein activity is about 70% to about 125% of the original activity. In some embodiments, the liquid pharmaceutical composition disclosed herein maintains stability as assessed by activation of IL-15 receptor on U2OS IL2Rβ/IL2Rγ cells upon storage at about 2° C. to about 8° C. for at least about 6 months, at least about 12 months or at least about 24 months, wherein activity is about 80% to about 125% of the original activity.

In some embodiments, the liquid pharmaceutical composition disclosed herein maintains biological activity as assessed by activation of IL-15 receptor on U2OS IL2Rβ/IL2Rγ cells upon storage at about 25° C. for at least about 1 month, for at least about 2 months, for at least about 3 months, for at least about 4 months, for at least about 5 months, for at least about 6 months or at least about 12 months, wherein activity is about 70% to about 125% of the original activity. In some embodiments, the liquid pharmaceutical composition disclosed herein maintains stability as assessed by activation of IL-15 receptor on U2OS IL2Rβ/IL2Rγ cells upon storage at about 25° C. for at least about 1 month, for at least about 2 months, for at least about 3 months, for at least about 4 months, for at least about 5 months, for at least about 6 months or at least about 12 months, wherein activity is about 80% to about 125% of the original activity.

In some embodiments, the liquid pharmaceutical composition disclosed herein maintains biological activity as assessed by activation of IL-15 receptor on U2OS IL2Rβ/IL2Rγ cells upon storage at about 40° C. for at least about 2 weeks, at least about 3 weeks, at least about 4 weeks (about 1 month), for at least about 2 months or for at least about 3 months, wherein activity is about 70% to about 125% of the original activity. In some embodiments, the liquid pharmaceutical composition disclosed herein maintains stability as assessed by activation of IL-15 receptor on U2OS IL2Rβ/IL2Rγ cells upon storage at about 40° C. for at least about 2 weeks, at least about 3 weeks, at least about 4 weeks (about 1 month), for at least about 2 months or for at least about 3 months, wherein activity is about 80% to about 125% of the original activity.

In some embodiments, the solid pharmaceutical composition disclosed herein maintains biological activity as assessed by activation of IL-15 receptor on U2OS IL2Rβ/IL2Rγ cells upon storage at about 2° C. to about 8° C. for at least about 6 months, at least about 12 months or at least about 24 months, wherein activity is about 70% to about 125% of the original activity. In some embodiments, the solid pharmaceutical composition disclosed herein maintains stability as assessed by activation of IL-15 receptor on U2OS IL2Rβ/IL2Rγ cells upon storage at about 2° C. to about 8° C. for at least about 6 months, at least about 12 months or at least about 24 months, wherein activity is about 80% to about 125% of the original activity.

In some embodiments, the solid pharmaceutical composition disclosed herein maintains biological activity as assessed by activation of IL-15 receptor on U2OS IL2Rβ/IL2Rγ cells upon storage at about 25° C. for at least about 1 month, for at least about 2 months, for at least about 3 months, for at least about 4 months, for at least about 5 months, for at least about 6 months or at least about 12 months, wherein activity is about 70% to about 125% of the original activity. In some embodiments, the solid pharmaceutical composition disclosed herein maintains stability as assessed by activation of IL-15 receptor on U2OS IL2Rβ/IL2Rγ cells upon storage at about 25° C. for at least about 1 month, for at least about 2 months, for at least about 3 months, for at least about 4 months, for at least about 5 months, for at least about 6 months or at least about 12 months, wherein activity is about 80% to about 125% of the original activity.

In some embodiments, the solid pharmaceutical composition disclosed herein maintains biological activity as assessed by activation of IL-15 receptor on U2OS IL2Rβ/IL2Rγ cells upon storage at about 40° C. for at least about 2 weeks, at least about 3 weeks, at least about 4 weeks (about 1 month), for at least about 2 months or for at least about 3 months, wherein activity is about 70% to about 125% of the original activity. In some embodiments, the solid pharmaceutical composition disclosed herein maintains stability as assessed by activation of IL-15 receptor on U2OS IL2Rβ/IL2Rγ cells upon storage at about 40° C. for at least about 2 weeks, at least about 3 weeks, at least about 4 weeks (about 1 month), for at least about 2 months or for at least about 3 months, wherein activity is about 80% to about 125% of the original activity.

### IL-15/IL-15Rα Concentration

The IL-15/IL-15Rα complex (e.g. as disclosed herein) used in the disclosed pharmaceutical compositions are described herein. In one embodiment, the IL-15/IL-15Rα complex comprises IL-15 comprising SEQ ID NO: 2 and IL-15Rα comprising SEQ ID NO: 5. In another embodiment, the IL-15/IL-15Rα complex comprises IL-15 consisting of SEQ ID NO: 2 and IL-15Rα consisting of SEQ ID NO: 5.

In some embodiments, the concentration of the IL-15/IL-15Rα protein complex is from about 0.1 mg/mL to about 50 mg/mL in the pharmaceutical composition. In one embodiment, the concentration of the IL-15/IL-15Rα protein complex is from about 0.1 mg/mL to about 20 mg/mL in the pharmaceutical composition. It is preferably from about 0.1 mg/mL to about 20 mg/mL, most preferably from about 0.1 mg/mL to about 10 mg/mL. Non-limiting examples include about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL.

The liquid and/or solid pharmaceutical compositions of the disclosure may include one or more stabilizers, wherein non-ionic stabilizers are preferred. Suitable non-ionic stabilizers include polyols or sugars such as monosaccharides, disaccharides or trisaccharides, e.g. sucrose, trehalose, raffinose, maltose, sorbitol or mannitol. The sugar may be a sugar alcohol or an amino sugar. In a preferred embodiment, the non-ionic stabilizer is a polyol or a sugar. In a preferred embodiment, the non-ionic stabilizer is sucrose, trehalose, glycerol, mannitol or sorbitol. In another preferred embodiment, the non-ionic stabilizer is sucrose. The concentration of the non-ionic stabilizer may be about 50 mM to about 500 mM, e.g. about 120 mM to about 350 mM, e.g. about 175 mM to about 350 mM, e.g. about 180 mM to about 300 mM, e.g. about 200 mM to about 300 mM, e.g. about 220 mM to about 300 mM, e.g. about 250 mM to about 270 mM, about 175 mM, about 180 mM, about 185 mM, about 190 mM, about 195 mM, about 200 mM, about 205 mM, about 210 mM, about 215 mM, about 220 mM, about 225 mM, about 230 mM, about 235 mM, about 240 mM, about 245 mM, about 250 mM, about 255 mM, about 260 mM, about 265 mM, about 270 mM, about 275 mM, about 280 mM, about 285 mM, about 290 mM, about 295 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM. In a preferred embodiment, the concentration of the non-ionic stabilizer in the stable liquid pharmaceutical composition is about 120 mM to about 350 mM. In another preferred embodiment, the concentration of the non-ionic stabilizer in the stable liquid pharmaceutical composition is about 180 mM to about 300 mM. In yet another preferred embodiment, the non-ionic stabilizer is sucrose, trehalose, glycerol, mannitol or sorbitol, wherein the concentration of the non-ionic stabilizer is about 120 mM to about 350 mM. In yet another preferred embodiment, the non-ionic stabilizer is sucrose, trehalose, glycerol, mannitol or sorbitol, wherein the concentration of the non-ionic stabilizer is about 180 mM to about 300 mM. In another preferred embodiment, the non-ionic stabilizer is mannitol, wherein the concentration of mannitol is about 120 mM to about 350 mM. In another preferred embodiment, the non-ionic stabilizer is mannitol, wherein the concentration of mannitol is about 180 mM to about 300 mM. In yet another embodiment, the non-ionic stabilizer is mannitol, wherein the concentration of mannitol is about 260 mM. In another preferred embodiment, the non-ionic stabilizer is sucrose, wherein the concentration of sucrose is about 120 mM to about 350 mM. In another preferred embodiment, the non-ionic stabilizer is sucrose, wherein the concentration of sucrose is about 180 mM to about 300 mM. In yet another embodiment, the non-ionic stabilizer is sucrose, wherein the concentration of sucrose is about 260 mM.

### Other Excipients

The liquid and/or solid pharmaceutical compositions provided herein may include further excipients, e.g. additional buffers, salts (e.g. sodium chloride, sodium succinate, sodium sulfate, potassium chloride, magnesium chloride, magnesium sulfate, and calcium chloride), additional stabilizing agents, tonicity modifier (e.g. salts and amino acids [e.g. proline, alanine, L-arginine, asparagine, L-aspartic acid, glycine, serine, lysine, and histidine]), glycerol, albumin, alcohols, preservatives, additional surfactants, anti-oxidants, etc. The liquid and/or solid pharmaceutical compositions may also comprise one or more tonicity agents. The term “tonicity agents” denotes pharmaceutically acceptable excipients used to modulate the tonicity of the liquid and/or solid pharmaceutical compositions. The liquid and/or solid pharmaceutical compositions can be hypotonic, isotonic or hypertonic. Isotonicity in general relates to the osmotic pressure of a solution, usually relative to that of human blood serum (around 250-350 mOsmol/kg). The liquid and/or solid pharmaceutical compositions described herein can be hypotonic, isotonic or hypertonic but will preferably be isotonic. An isotonic formulation denotes a solution having the same tonicity as some other solution with which it is compared, such as physiologic salt solution and the blood serum. Suitable tonicity agents comprise but are not limited to sodium chloride, potassium chloride, glycerin and any component from the group of amino acids or sugars, in particular glucose. Tonicity agents are generally used in an amount of about 0.1 mM to about 500 mM.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is sucrose. A thorough discussion of such additional pharmaceutical ingredients is available in Gennaro (2000) Remington: The Science and Practice of Pharmacy. 20th edition, ISBN: 0683306472.

### Liquid Pharmaceutical Compositions

Disclosed herein are stable liquid pharmaceutical compositions comprising a heterodimeric IL-15/IL-15Rα complex, e.g. as described herein, and about 0.0001% to about 1% (w/v) of a surfactant, optionally further comprising about 1 mM to about 100 mM of a buffering agent providing a pH in the range of from about 4.5 to about 8.5, optionally further comprising about 1 mM to about 500 mM of at least one stabilizer described supra. Preferred heterodimeric IL-15/IL-15Rα complex that may be comprised in the stable liquid pharmaceutical composition are described in detail herein. Particularly preferred is the IL-15/IL-15Rα complex comprising IL-15 comprising SEQ ID NO: 2 and IL-15Rα comprising SEQ ID NO: 5 as disclosed herein.

Suitable surfactants for use with the disclosed stable liquid pharmaceutical compositions include, but are not limited to, non-ionic surfactants, ionic surfactants, zwitterionic surfactants and combinations thereof. Typical surfactants for use include, but are not limited to, sorbitan fatty acid esters (e.g. sorbitan monocaprylate, sorbitan monolaurate, sorbitan monopalmitate), sorbitan trioleate, glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearic acid amide); C10-C18 alkyl sulfates (e.g. sodium cetyl sulfate, sodium lauryl sulfate, sodium oleyl sulfate), polyoxyethylene C10-C18 alkyl ether sulfate with an average of 2 to 4 moles of ethylene oxide units added (e.g. sodium polyoxyethylene lauryl sulfate), and C1-C18 alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C12-C18 fatty acids. A composition may include one or more of these surfactants. Preferred surfactants are poloxamer (e.g. Poloxamer 188, Poloxamer 407 , poloxamer 403 , poloxamer 402 , poloxamer 181 , poloxamer 401 , poloxamer 185 , and poloxamer 338 or polyoxyethylene sorbitan fatty acid esters, e.g. polysorbate 20, 40, 60 or 80. Polysorbate 20 (Tween 20) (e.g. at a concentration of about 0.01% to about 0.1% (w/v), e.g. about 0.01% to about 0.04% (w/v), e.g. about 0.01%, about 0.02%, about 0.04%, about 0.06%, about 0.08%, about 0.1%) is useful. Polysorbate 80 (Tween 80) (e.g. at a concentration of about 0.01% to about 0.1% (w/v), e.g. about 0.01% to about 0.04% (w/v), e.g. about 0.01%, about 0.02%, about 0.04%, about 0.06%, about 0.08%, about 0.1%) is useful. Poloxamer 188 (e.g. at a concentration of about 0.01% to about 1% (w/v), e.g. about 0.1% to about 0.5% (w/v), e.g. about 0.1%, about 0.2%, about 0.3%, about 0.4%, about 0.5%, about 0.6%, about 0.7%, about 0.8%, about 0.9%, about 1%) is particularly useful. In one embodiment, the stable liquid pharmaceutical composition comprises about 0.2% (w/v) Poloxamer 188. In an alternative embodiment, the surfactant comprised in the stable liquid pharmaceutical composition is a polysorbate, suitably polysorbate 20 or polysorbate 80, suitably polysorbate 20. Suitably, the polysorbate is at a concentration of about 0.01% to about 0.1% (w/v), suitably about 0.02% to about 0.05% (w/v), suitably about 0.04% (w/v).

In one embodiment, the concentration of the heterodimeric IL-15/IL-15Rα complex comprised in the stable liquid pharmaceutical composition is in the range of about 0.1 mg/mL to about 50 mg/mL, more preferred about 0.1 mg/mL to about 10 mg/mL, most preferred about 1 mg/m L.

In certain embodiments, the buffering agent comprised in the stable liquid pharmaceutical composition is acetate buffer, succinate buffer, citrate buffer or histidine buffer. Particularly preferred is a L-histidine/HCl buffer (i.e., L-histidine as the buffering agent). Acetate buffer, in particular sodium acetate buffer was assessed as beneficial in the liquid pharmaceutical compositions with regard to degradation products by SEC, AEX—and aggregation products by RP-HPLC. In one embodiment, the stable liquid composition comprises about 10 mM to about 50 mM, e.g. about 10 mM, e.g. about 15 mM, e.g. about 20 mM, e.g. about 25 mM, e.g. about 30 mM, e.g. about 35 mM, e.g. about 40 mM, e.g. about 45 mM, e.g. about 50 mM, Na-acetate buffer. In another embodiment, the stable liquid composition comprises about 15 mM to about 30 mM Na-acetate buffer. In another embodiment, the stable liquid composition comprises about 20 mM to about 30 mM Na-acetate buffer. In another embodiment, the stable liquid composition comprises about 10 mM to about 30 mM Na-acetate buffer. In one embodiment, the stable liquid composition comprises about 10 mM to about 50 mM, e.g. about 10 mM, e.g. about 15 mM, e.g. about 20 mM, e.g. about 25 mM, e.g. about 30 mM, e.g. about 35 mM, e.g. about 40 mM, e.g. about 45 mM, e.g. about 50 mM, histidine buffer. In another embodiment, the stable liquid composition comprises about 15 mM to about 30 mM histidine buffer. In another embodiment, the stable liquid composition comprises about 20 mM to about 30 mM histidine buffer. In another embodiment, the stable liquid composition comprises about 10 mM to about 30 mM histidine buffer.

In one embodiment, the pH of the stable liquid pharmaceutical composition is in the range of about 4.5 to about 8.5, e.g. about 4.5 to about 7.5, e.g. about 4.5 to about 6.5, e.g. about 4.5 to about 5.5, e.g. about 4.7 to about 5.5, e.g. about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6, about 6.2, about 6.4, about 6.5, about 6.6, about 6.7, about 6.8, about 6.9, about 7.0, about 7.1, about 7.2, about 7.3, about 7.4, about 7.5. In a preferred embodiment the pH of the stable liquid composition is in the range of about 4.5 to about 5.5. Overall testing indicated that the ideal composition pH of the disclosed liquid pharmaceutical composition is about 5.0. Thus, in one embodiment, the pH of the stable liquid pharmaceutical composition is about 5.0. In one embodiment, the stable liquid pharmaceutical composition comprises about 10 mM to about 50 mM Na-acetate buffer at pH of about 4.5 to about 8.5. In another embodiment, the stable liquid pharmaceutical composition comprises about 15 mM to about 30 mM Na-acetate buffer at pH of about 4.5 to about 8.5. In another embodiment, the stable liquid pharmaceutical composition comprises about 20 mM to about 30 mM Na-acetate buffer at pH of about 4.5 to about 8.5. In another embodiment, the stable liquid pharmaceutical composition comprises about 10 mM to about 30 mM Na-acetate buffer at pH of about 4.5 to about 8.5. In another embodiment, the stable liquid pharmaceutical composition comprises about 10 mM to about 50 mM Na-acetate buffer at pH of about 4.5 to about 5.5. In another embodiment, the stable liquid pharmaceutical composition comprises about 15 mM to about 30 mM Na-acetate buffer at pH of about 4.5 to about 5.5. In another embodiment, the stable liquid pharmaceutical composition comprises about 20 mM to about 30 mM Na-acetate buffer at pH of about 4.5 to about 5.5. In another embodiment, the stable liquid pharmaceutical composition comprises about 10 mM to about 30 mM Na-acetate buffer at pH of about 4.5 to about 5.5. In another embodiment, the stable liquid pharmaceutical composition comprises about 10 mM to about 50 mM Na-acetate buffer at pH of about 5.0. In another embodiment, the stable liquid pharmaceutical composition comprises about 15 mM to about 30 mM Na-acetate buffer at pH of about 5.0. In another embodiment, the stable liquid pharmaceutical composition comprises about 20 mM to about 30 mM Na-acetate buffer at pH of about 5.0. In another embodiment, the stable liquid pharmaceutical composition comprises about 10 mM to about 30 mM Na-acetate buffer at pH of about 5.0. In another embodiment, the stable liquid pharmaceutical composition comprises about 20 mM Na-acetate buffer at pH of about 5.0. In one embodiment, the stable liquid pharmaceutical composition comprises about 10 mM to about 50 mM histidine buffer at pH of about 4.5 to about 8.5. In another embodiment, the stable liquid pharmaceutical composition comprises about 15 mM to about 30 mM histidine buffer at pH of about 4.5 to about 8.5. In another embodiment, the stable liquid pharmaceutical composition comprises about 20 mM to about 30 mM histidine buffer at pH of about 4.5 to about 8.5. In another embodiment, the stable liquid pharmaceutical composition comprises about 10 mM to about 30 mM histidine buffer at pH of about 4.5 to about 8.5. In another embodiment, the stable liquid pharmaceutical composition comprises about 10 mM to about 50 mM histidine buffer at pH of about 4.5 to about 5.5. In another embodiment, the stable liquid pharmaceutical composition comprises about 15 mM to about 30 mM histidine buffer at pH of about 4.5 to about 5.5. In another embodiment, the stable liquid pharmaceutical composition comprises about 20 mM to about 30 mM histidine buffer at pH of about 4.5 to about 5.5. In another embodiment, the stable liquid pharmaceutical composition comprises about 10 mM to about 30 mM histidine buffer at pH of about 4.5 to about 5.5. In another embodiment, the stable liquid pharmaceutical composition comprises about 10 mM to about 50 mM histidine buffer at pH of about 5.0. In another embodiment, the stable liquid pharmaceutical composition comprises about 15 mM to about 30 mM histidine buffer at pH of about 5.0. In another embodiment, the stable liquid pharmaceutical composition comprises about 20 mM to about 30 mM histidine buffer at pH of about 5.0. In another embodiment, the stable liquid pharmaceutical composition comprises about 10 mM to about 30 mM histidine buffer at pH of about 5.0. In another embodiment, the stable liquid pharmaceutical composition comprises about 20 mM histidine buffer at pH of about 5.0.

In particular embodiments, the stable liquid pharmaceutical composition comprises
a. About 1 mg/mL of IL-15/IL-15 Rα complex as described herein, about 20 mM sodium acetate, about 260 mM sucrose, about 0.2% Poloxamer 188, wherein the pH of the composition is about 4.7.
b. About 1 mg/mL of IL-15/IL-15 Rα complex as described herein, about 20 mM sodium acetate, about 260 mM sucrose, about 0.04% Polysorbate 20, wherein the pH of the composition is about 4.7.
c. About 1 mg/mL of IL-15/IL-15 Rα complex as described herein, about 20 mM sodium acetate, about 260 mM sucrose, about 0.2% Poloxamer 188, wherein the pH of the composition is about 5.
d. About 1 mg/mL of IL-15/IL-15 Rα complex as described herein, about 20 mM sodium acetate, about 260 mM sucrose, about 0.04% Polysorbate 20, wherein the pH of the composition is about 5.
e. About 1 mg/mL of IL-15/IL-15 Rα complex as described herein, about 20 mM sodium acetate, about 260 mM sucrose, about 0.2% Poloxamer 188, wherein the pH of the composition is about 5.5.
f. About 1 mg/mL of IL-15/IL-15 Rα complex as described herein, about 20 mM sodium acetate, about 260 mM sucrose, about 0.04% Polysorbate 20, wherein the pH of the composition is about 5.5.
g. About 1 mg/mL of IL-15/IL-15 Rα complex as described herein, about 20 mM sodium acetate, about 260 mM sucrose, about 0.2% Poloxamer 188, wherein the pH of the composition is about 4.7.
h. About 1 mg/mL of IL-15/IL-15 Rα complex as described herein, about 20 mM histidine, about 260 mM sucrose, about 0.04% Polysorbate 20, wherein the pH of the composition is about 4.7.
i. About 1 mg/mL of IL-15/IL-15 Rα complex as described herein, about 20 mM histidine, about 260 mM sucrose, about 0.2% Poloxamer 188, wherein the pH of the composition is about 5.
j. About 1 mg/mL of IL-15/IL-15 Rα complex as described herein, about 20 mM histidine, about 260 mM sucrose, about 0.04% Polysorbate 20, wherein the pH of the composition is about 5.
k. About 1 mg/mL of IL-15/IL-15 Rα complex as described herein, about 20 mM histidine, about 260 mM sucrose, about 0.2% Poloxamer 188, wherein the pH of the composition is about 5.5or
l. About 1 mg/mL of IL-15/IL-15 Rα complex as described herein, about 20 mM histidine, about 260 mM sucrose, about 0.04% Polysorbate 20, wherein the pH of the compositions is about 5.5.

Solid Pharmaceutical Compositions

Also disclosed herein are solid pharmaceutical compositions comprising a heterodimeric IL-15/IL-15Rα complex, e.g. as described herein; and comprising about 10 mM to about 50 mM of a buffering agent providing a pH in the range of from about 6.5 to about 8.5, about 1 mM to about 500 mM of at least one stabilizer described supra and about 0.1 mM to about 50 mM of at least one tonicity agent described supra.

Solid formulations of the invention are generally prepared by drying a liquid formulation. Any suitable method of drying can be used, such as lyophilization or spray drying. In one aspect, a lyoprotectant is added to the formulation prior to lyophilization. Lyophilization involves freezing a liquid formulation, usually in the container that will be used to store, ship and distribute the formulation (e.g. a vial, syringe (e.g. a single- or dual-chamber syringe), or cartridge (e.g. a single- or dual-chamber cartridge) (See, e.g. Gatlin and Nail in Protein Purification Process Engineering, ed. Roger G. Harrison, Marcel Dekker Inc., 317-367 (1994). Once the formulation is frozen, the atmospheric pressure is reduced and the temperature is adjusted to allow removal of the frozen solvent e.g. through sublimation. This step of the lyophilization process is sometimes referred to as primary drying. If desired, the temperature can then be raised to remove any solvent that is still bound to the dry formulation by evaporation. This step of the lyophilization process is sometimes referred to as secondary drying. When the formulation has reached the desired degree of dryness, the drying process is concluded and the containers are sealed. The final solid formulation is sometimes referred to as a “lyophilized formulation” or a “cake.” The lyophilization process can be performed using any suitable equipment. Suitable lyophilization equipment is available from a number of commercial sources (e.g. SP Scientific, Stone Ridge, N.Y.).

A variety of suitable apparatuses can be used to dry liquid formulations to produce a solid (e.g. lyophilized) formulation. Generally, lyophilized formulations are prepared by those of skill in the art using a sealed chamber that contains shelves, on which vials of the liquid formulation to be dried are placed. The temperature of the shelves, as well as cooling and heating rate can be controlled, as can the pressure inside the chamber. It will be understood that various process parameters discussed herein refer to processes performed using this type of apparatus. Persons of ordinary skill can easily adapt the parameters described herein to other types of drying apparatuses if desired.

Suitable temperatures and the amount of vacuum for primary and secondary drying can be readily determined by a person of ordinary skill. In general, the formulation is frozen at a temperature of about −30° C. or less, such as −40° C. or −50° C. The rate of cooling can affect the amount and size of ice crystals in the matrix. Primary drying is generally conducted at a temperature that is about 10° C., about 20° C., about 30° C., about 40° C. or about 50° C. warmer than the freezing temperature.

After lyophilization, the vial, syringe, or cartridge can be sealed, e.g. stoppered, under a vacuum. Alternatively, a gas, e.g. dry air or nitrogen, can be allowed into the container prior to sealing. Where oxidation is a concern, the gas allowed into the lyophilization chamber can comprise a gas which retards or prevents oxidation of the lyophilized product. The gases can be non-oxygenated gases, e.g. nitrogen, or can be an inert gas, e.g. helium, neon, argon, krypton or xenon.

Preferred heterodimeric IL-15/IL-15Rα complex that may be comprised in the solid pharmaceutical composition are described in detail herein. Particularly preferred is the IL-15/IL-15Rα complex comprising IL-15 comprising SEQ ID NO: 2 and IL-15Rα comprising SEQ ID NO: 5 as disclosed herein. Particularly preferred is the IL-15/IL-15Rα complex comprising IL-15 consisting of SEQ ID NO: 2 and IL-15Rα consisting of SEQ ID NO: 5 as disclosed herein.

In one embodiment, the concentration of the heterodimeric IL-15/IL-15Rα complex comprised in the solid pharmaceutical composition is in the range of about 0.1 mg/mL to about 50 mg/mL, more preferred about 0.1 mg/mL to about 10 mg/mL, most preferred about 0.1 mg/mL to about 0.5 mg/mL.

In certain embodiments, the buffering agent comprised in the solid pharmaceutical composition is phosphate buffer, acetate buffer, succinate buffer, citrate buffer or histidine buffer. Particularly preferred is a Na/K phosphate buffer. In one embodiment, the solid composition comprises about 10 mM to about 50 mM, e.g. about 10 mM, e.g. about 15 mM, e.g. about 20 mM, e.g. about 25 mM, e.g. about 30 mM, e.g. about 35 mM, e.g. about 40 mM, e.g. about 45 mM, e.g. about 50 mM Na/K phosphate buffer buffer. In another embodiment, the solid composition comprises about 15 mM to about 30 mM Na/K phosphate buffer. In another embodiment, the solid composition comprises about 20 mM to about 30 mM Na/K phosphate buffer. In another embodiment, the solid composition comprises about 10 mM to about 30 mM Na/K phosphate buffer.

In one embodiment, the pH of the solid pharmaceutical composition is in the range of about 6.5 to about 8.5, e.g. about 6.5 to about 8, e.g. about 6.5 to about 7.5, e.g. about 6.8 to about 7.5, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5. In a preferred embodiment the pH of the solid composition is in the range of about 6.5 to about 7.5. Overall testing indicated that the ideal composition pH of the disclosed solid pharmaceutical composition is about 7.3. Thus, in one embodiment, the pH of the solid pharmaceutical composition is about 7.3. In one embodiment, the solid pharmaceutical composition comprises about 1 mM to about 50 mM Na/K phosphate buffer at pH of about 6.5 to about 8.5. In another embodiment, the solid pharmaceutical composition comprises about 1 mM to about 30 mM Na/K phosphate buffer at pH of about 6.5 to about 8.5. In another embodiment, the solid pharmaceutical composition comprises about 1 mM to about 10 mM Na/K phosphate buffer at pH of about 6.5 to about 8.5. In another embodiment, the solid pharmaceutical composition comprises about 1 mM to about 50 mM Na/K phosphate buffer at pH of about 6.5 to about 7.5. In another embodiment, the solid pharmaceutical composition comprises about 1 mM to about 30 mM Na/K phosphate buffer at pH of about 6.5 to about 7.5. In another embodiment, the solid pharmaceutical composition comprises about 1 mM to about 10 mM Na/K phosphate buffer at pH of about 6.5 to about 7.5. In another embodiment, the solid pharmaceutical composition comprises about 1 mM to about 50 mM Na/K phosphate buffer at pH of about 7.3. In another embodiment, the solid pharmaceutical composition comprises about 1 mM to about 30 mM Na/K phosphate buffer at pH of about 7.3. In another embodiment, the solid pharmaceutical composition comprises about 1 mM to about 10 mM Na/K phosphate buffer at pH of about 7.3. In another embodiment, the solid pharmaceutical composition comprises about 1 mM to about 5 mM Na/K phosphate buffer at pH of about 7.3. In another embodiment, the solid pharmaceutical composition comprises about 1.35 mM Na/K phosphate buffer at pH of about 7.3.

The solid pharmaceutical composition also comprises about 1 mM to about 500 mM of at least one stabilizer. In a preferred embodiment, the solid pharmaceutical composition comprises about 1 mM to about 500 mM of at least two stabilizers. Suitable stabilizers are e.g. described supra. In one embodiment, the solid pharmaceutical composition comprises about 1 mM to about 500 mM sucrose and about 1 mM to about 500 mM mannitol. In another embodiment, the solid pharmaceutical composition comprises about 5 mM to about 50 mM sucrose and about 100 mM to about 300 mM mannitol. In another embodiment, the solid pharmaceutical composition comprises about 30 mM sucrose and about 220 mM mannitol.

The solid pharmaceutical composition also comprises about 0.1 mM to about 50 mM of at least one tonicity agent. In a preferred embodiment, the solid pharmaceutical composition comprises about 0.1 mM to about 50 mM of at least two tonicity agents. Suitable tonicity agents are e.g. described supra. In one embodiment, the solid pharmaceutical composition comprises about 0.1 mM to about 50 mM KCl and about 0.1 mM to about 50 mM NaCl. In another embodiment, the solid pharmaceutical composition comprises about 0.1 mM to about 1 mM KCl and about 10 mM to about 50 mM NaCl. In another embodiment, the solid pharmaceutical composition comprises about 0.375 mM KCl and about 20 mM NaCl.

In one embodiment, the solid pharmaceutical composition comprises about 0.24 mg/mL IL-15/IL-15Rα complex, about 30 mM sucrose, about 220 mM mannitol, about 0.375 mM KCl, about 20 mM NaCl, and about 1.35 mM Na/K phosphate buffer at about pH7.3.

### Articles of Manufacture

In another aspect, provided herein is an article of manufacture which contains the pharmaceutical formulation presently disclosed and provides instructions for its use. The article of manufacture comprises a container. Suitable containers include, for example, bottles, vials (e.g. dual chamber vials, a vial of liquid formulation with or without a needle, a vial of solid formulation with or without a vial of reconstitution liquid with or without a needle), syringes (such as dual chamber syringes, preloaded/prefilled syringes (e.g. for use in an auto-injector device), an auto-injector), cartridges, pens and test tubes. The container can be formed from a variety of materials such as glass, metal or plastic. The container holds the formulation and a label on, or associated with, the container can indicate directions for use. In another embodiment, the formulation can be prepared for self-administration and/or contain instructions for self-administration. In one embodiment, the container holding the formulation can be a single-use vial. In another embodiment, the container holding the formulation can be a multi-use vial, which allows for repeat administration of the formulation, e.g. using more than one portion of a reconstituted formulation. The article of manufacture can further include other materials desirable from a commercial and user standpoint, including other buffers, diluents, filters, needles, syringes and package inserts with instructions for use as noted in the previous section.

In one aspect, provided is an article of manufacture comprising: a container and a liquid pharmaceutical composition disposed within said container, said composition comprising a heterodimeric IL-15/IL-15Rα complex (e.g. about 0.1 mg/mL to about 50 mg/mL or about 0.1 to about 10 mg/mL); and about 0.0001% to about 1% (w/v) of a surfactant, optionally further comprising about 1 mM to about 100 mM of a buffering agent providing a pH in the range of from about 4.5 to about 8.5, optionally further comprising about 1 mM to about 500 mM of at least one stabilizer, wherein the liquid pharmaceutical composition is not reconstituted from a lyophilizate.

In another aspect, provided is an article of manufacture comprising: a container and a solid pharmaceutical composition disposed within said container, said composition comprising a heterodimeric IL-15/IL-15Rα complex (e.g. about 0.1 mg/mL to about 50 mg/mL or about 0.1 mg/mL to about 10 mg/mL); and about 10 mM to about 50 mM of a buffering agent providing a pH in the range of from about 6.5 to about 8.5, about 1 mM to about 500 mM of at least one stabilizer and about 0.1 mM to about 50 mM of at least one tonicity agent. In one embodiment, the composition is lyophilized and stored as a single dose in one container. The container can be stored at about 2-8° C. or 25 ° C. until it is administered to a subject in need thereof.

In some embodiments, the liquid or solid pharmaceutical composition has a sufficient amount of the heterodimeric IL-15/IL-15Rα complex to allow delivery of at least about 0.1 to about 10 μg/kg heterodimeric IL-15/IL-15Rα complex (e.g. as disclosed herein) per unit dose. In some embodiments, the liquid or solid pharmaceutical product has a sufficient amount of the heterodimeric IL-15/IL-15Rα complex (e.g. as disclosed herein) to allow delivery of at least about 0.1 μg/kg, about 0.25 μg/kg, about 0.5 μg/kg, about 1 μg/kg, about 2 μg/kg or about 5 pg/kg per unit dose. In some embodiments, the liquid or solid pharmaceutical product is formulated at a dosage to allow subcutaneous delivery of about 0.1 μg/kg to about 10 μg/kg heterodimeric IL-15/IL-15Rα complex (e.g. as disclosed herein) per unit dose. In some embodiments, the liquid or solid pharmaceutical composition is formulated at a dosage to allow intravenous delivery of about 0.1 μg/kg to about 10 μg/kg heterodimeric IL-15/IL-15Rα complex (e.g. as disclosed herein) per unit dose.

### Kits Comprising Pharmaceutical Products and Compositions

The disclosure also encompasses kits for treating a patient. Such kits broadly include at least one of the disclosed pharmaceutical products or liquid or solid compositions and instructions for use. The instructions will disclose appropriate techniques for the provision of the pharmaceutical composition to the patient as part of a dosing regimen. These kits may also contain additional agents for treatment for delivery in combination with (i.e., simultaneously or sequentially [before or after]) the enclosed pharmaceutical composition.

Disclosed herein are kits for the treatment of a patient in need thereof, comprising: a) a container, b) a liquid pharmaceutical composition disposed within said container, said composition comprising: i) a heterodimeric IL-15/IL-15Rα complex (e.g. about 0.1 mg/mL to about 50 mg/mL or about 0.1 mg/mL to about 10 mg/mL); and about 0.0001% to about 1% (w/v) of a surfactant, optionally further comprising about 1 mM to about 100 mM of a buffering agent providing a pH in the range of from about 4.5 to about 8.5, optionally further comprising about 1 mM to about 500 mM of at least one stabilizer, wherein the liquid pharmaceutical composition is not reconstituted from a lyophilisate; and c) instructions for administering the liquid pharmaceutical composition to the patient. In some embodiments, the container is a pen, pre-filled syringe, autoinjector or vial. In one embodiment, the container is a syringe. The syringe may be comprised in an autoinjector. In another embodiment, the container is an autoinjector comprising the liquid formulation described herein.

Disclosed herein are kits for the treatment of a patient in need thereof, comprising: a) a container, b) a solid pharmaceutical composition disposed within said container, said composition comprising: i) a heterodimeric IL-15/IL-15Rα complex (e.g. about 0.1 mg/mL to about 0.5 mg/mL), about 10 mM to about 50 mM of a buffering agent providing a pH in the range of from about 6.5 to about 8.5, about 1 mM to about 500 mM of at least one stabilizer and about 0.1 mM to about 50 mM of at least one tonicity agent; and c) instructions for administering the liquid pharmaceutical composition to the patient. In some embodiments, the container is a pen, pre-filled syringe, autoinjector or vial. In one embodiment, the container is a syringe. The syringe may be comprised in an autoinjector. In another embodiment, the container is an autoinjector comprising the solid formulation described herein.

### Methods of Using Pharmaceutical Products and Compositions

The disclosed pharmaceutical compositions are used for the treatment of patients that benefit from treatment with an IL-15/IL-15Rα complex, e.g. as described herein. The appropriate dosage will, of course, vary depending upon, for example, the particular IL-15/IL-15Rα complex, e.g. as disclosed herein, to be employed, the host, the mode of administration and the nature and severity of the condition being treated, and on the nature of prior treatments that the patient has undergone. Ultimately, the attending health care provider will decide the amount of the IL-15/IL-15Rα complex with which to treat each individual patient.

Provided herein are methods of treatment, comprising administering to a patient in need thereof, e.g. as described herein, a therapeutically effective dose of an IL-15/IL-15Rα complex, e.g. as disclosed herein, e.g. by subcutaneous injection, wherein the IL-15/IL-15Rα complex is provided as part of a pharmaceutical composition as described herein, e.g. as a liquid pharmaceutical composition or as a solid pharmaceutical composition as described herein.

Also provided herein are pharmaceutical compositions, e.g. as disclosed herein, for use in the treatment of a patient in need thereof, e.g. as described herein, comprising administering to the patient a therapeutically effective dose of an IL-15/IL-1 5Rα complex, e.g. as disclosed herein, e.g. by subcutaneous injection, wherein the IL-15/IL-15Rα complex is provided as part of a pharmaceutical composition as described herein, e.g. as a liquid pharmaceutical composition or as a solid pharmaceutical composition as described herein.

Also provided herein is the use of an IL-15/IL-15Rα complex, e.g. as disclosed herein, for the manufacture of a medicament for the treatment of a patient in need thereof, e.g. as described herein, comprising administering to the patient a therapeutically effective dose of an IL-15/IL-15Rα complex, e.g. as disclosed herein, e.g. by subcutaneous injection, wherein the IL-15/IL-15Rα complex is provided as part of a pharmaceutical composition as described herein, e.g. as a liquid pharmaceutical composition or as a solid pharmaceutical composition as described herein.

In one aspect, provided herein are methods for enhancing IL-15-mediated immune function, comprising administering to subjects IL-15/IL-15Rα complex, e.g. as disclosed herein, in a specific dose regimen, wherein the IL-15/IL-15Rα complex is provided as part of a pharmaceutical composition as described herein, e.g. as a liquid pharmaceutical composition or as a solid pharmaceutical composition as described herein. Since enhancing IL-15-mediated immune function is beneficial for the prevention, treatment and/or management of certain disorders, provided herein are methods for the prevention, treatment and/or management of such disorders comprising administering to a subject in need thereof an IL-15/IL-15Rα complex, e.g. as disclosed herein, wherein the IL-15/IL-15Rα complex is provided as part of a pharmaceutical composition as described herein, e.g. as a liquid pharmaceutical composition or as a solid pharmaceutical composition as described herein. Non-limiting examples of disorders in which it is beneficial to enhance IL-15-mediated immune function include cancer, lymphopenia, immunodeficiencies, infectious diseases, and wounds.

In one embodiment, provided herein is a method for preventing, treating and/or managing disorders in a subject, wherein enhancement of IL-15-mediated immune function is beneficial for the prevention, treatment and/or management of such disorders, the method comprising administering the same dose of an IL-15/IL-15Rα complex, e.g. as disclosed herein, to a subject for the duration of the treatment cycle, wherein the IL-15/IL-15Rα complex is provided as part of a pharmaceutical composition as described herein, e.g. as a liquid pharmaceutical composition or as a solid pharmaceutical composition as described herein. In one embodiment, the dose is in the range of 0.1 μg/kg and 0.5 μg/kg. In one embodiment, the dose is in the range of 0.25 μg/kg and 1 μg/kg. In a specific embodiment, the dose is in the range of 0.5 μg/kg and 2 μg/kg. In another embodiment, the dose is between 1 μg/kg and 4 μg/kg. In another embodiment, the dose is between 2 μg/kg and 8 μg/kg. In another embodiment, the dose is 0.1 μg/kg, 0.25 μg/kg, 0.5 μg/kg, 1 μg/kg, 2 μg/kg, 4 μg/kg, 5 μg/kg, 6 μg/kg, 8 μg/kg. In a specific embodiment, the dose is 1 μg/kg. In certain embodiments, the dose is administered 1, 2, 3, 4, 5, 6, 7, 8, 9, 10 or more times, or 1 to 3, 1 to 4, 2 to 4, 2 to 5, 2 to 6, 3 to 6, 4 to 6, 6 to 8, 5 to 8, or 5 to 10 times. In some embodiments, the dose is administered 1, 2, 3, 4, 5, 6, 7, 8, 9, 10 or more times, or 1 to 3, 1 to 4, 2 to 4, 2 to 5, 1 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time. In specific embodiments, each dose is administered at least 1, 2, 3, 4, 5, 6 or more times over a 5 to 7 day, 5 to 10 day, 7 to 12 day, 7 to 14 day, 7 to 21 day or 14 to 21 day period of time. In another specific embodiment, each dose is administered at least once and the subject is administered a dose once per week for a three week period.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Rα complex, e.g. as disclosed herein, to the subject in a dosing regimen at least once, twice, four times or six times in a dosing cycle before a period of non-administration, wherein the IL-15/IL-15Rα complex is provided as part of a pharmaceutical composition as described herein, e.g. as a liquid pharmaceutical composition or as a solid pharmaceutical composition as described herein. In a specific embodiment the IL-15/IL-15Rα complex, e.g. as disclosed herein, is administered once a week for three weeks with no administration in week four, wherein the IL-15/IL-15Rα complex is provided as part of a pharmaceutical composition as described herein, e.g. as a liquid pharmaceutical composition or as a solid pharmaceutical composition as described herein. The dosing cycle is then repeated.

In an alternative embodiment, provided herein is a method for preventing, treating and/or managing disorders in a subject, wherein enhancement of IL-15-mediated immune function is beneficial for the prevention, treatment and/or management of such disorders, the method comprising (a) administering at least one initial low dose of an IL-15/IL-15Rα complex, e.g. as disclosed herein, to a subject; and (b) administering successively higher doses of the IL-15/IL-15Rα complex, e.g. as disclosed herein, to the subject for the duration of the treatment cycle, wherein the IL-15/IL-15Rα complex is provided as part of a pharmaceutical composition as described herein, e.g. as a liquid pharmaceutical composition or as a solid pharmaceutical composition as described herein. In a specific embodiment, provided herein is a method for preventing, treating and/or managing cancer in a subject, method comprising (a) administering an initial dose of an IL-15/IL-15Rα complex, e.g. as disclosed herein, to the subject for the duration of the treatment cycle; and (b) administering successively higher doses of the IL-15/IL-15Rα complex to the subject for the duration of the treatment cycle, wherein the IL-15/IL-15Rα complex is provided as part of a pharmaceutical composition as described herein, e.g. as a liquid pharmaceutical composition or as a solid pharmaceutical composition as described herein. In a specific embodiment, the initial dose is in the range of 0.1 μg/kg and 0.5 μg/kg. In a specific embodiment, the initial dose is in the range of 0.25 μg/kg and 1 μg/kg. In another embodiment, the initial dose is in the range of 0.5 μg/kg and 2 μg/kg. In a specific embodiment, the initial dose is between 1 μg/kg and 4 μg/kg. In another embodiment, the initial dose is between 2 μg/kg and 8 μg/kg. In another embodiment, the initial dose is about 0.25 μg/kg. In another embodiment, the initial dose is about 0.5 μg/kg. In another embodiment, the initial dose is about 1 μg/kg. In another embodiment, the initial dose is 0.1 μg/kg, 0.25 μg/kg, 0.5 μg/kg, 1 μg/kg, 2 μg/kg, 4 μg/kg, 5 μg/kg, 6 μg/kg, 8 μg/kg. In certain embodiments, the initial dose is administered 1, 2, 3, 4, 5, 6, 7, 8, 9, 10 or more times, or 1 to 3, 1 to 4, 2 to 4, 2 to 5, 2 to 6, 3 to 6, 4 to 6, 6 to 8, 5 to 8, or 5 to 10 times. In some embodiments, the initial dose is administered 1, 2, 3, 4, 5, 6, 7, 8, 9, 10 or more times, or 1 to 3, 1 to 4, 2 to 4, 2 to 5, 1 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time. In certain embodiments, each successively higher dose is 1.2, 1.25, 1.3, 1.35, 1.4, 1.45, 1.5, 2, 2.5, 3, 3.5, 4, 4.5, 5, 5.5, or 6 times higher than the previous dose, or 1.2 to 2, 2 to 3, 2 to 4, 1 to 5, 2 to 6, 3 to 4, 3 to 6, or 4 to 6 times higher than the previous dose, or 2 times higher than the previous dose. In some embodiments, each successively higher dose is 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% higher than the previous dose. In specific embodiments, each dose is administered at least 1, 2, 3, 4, 5, 6 or more times over a 5 to 7 day, 5 to 10 day, 7 to 12 day, 7 to 14 day, 7 to 21 day or 14 to 21 day period of time. In another specific embodiment, each dose is administered at least once and the subject is administered a dose three times per 7 day week (e.g. Monday, Wednesday and Friday) for a two week period.

In certain embodiments, the subject is monitored for the following adverse events, such as grade 3 or 4 thrombocytopenia, grade 3 or 4 granulocytopenia, grade 3 or 4 leukocytosis (White Blood Cell (WBC)>100,000 mm³), grade 3 or 4 decreases in WBC, absolute lymphocyte count (ALC) and/or absolute neutrophil count (ANC), lymphocytosis and organ dysfunction (e.g. liver or kidney dysfunction). In certain embodiments, the dose is not increased and the dose may be remain the same, be stopped or reduced if the subject experiences adverse events, such as grade 3 or 4 thrombocytopenia, grade 3 or 4 granulocytopenia, grade 3 or leukocytosis (White Blood Cell>100,000 mm³), grade 3 or 4 decreases in WBC, absolute lymphocyte count (ALC) and/or absolute neutrophil count (ANC), lymphocytosis, and organ dysfunction (e.g. liver or kidney dysfunction). In accordance with these embodiments, the dose of the IL-15/IL-15Rα complex, e.g. as disclosed herein, administered to the subject may be reduced or remain the same until the adverse events decrease or disappear, wherein the IL-15/IL-15Rα complex is provided as part of a pharmaceutical composition as described herein, e.g. as a liquid pharmaceutical composition or as a solid pharmaceutical composition as described herein.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Rα complex, e.g. as disclosed herein, to the human subject in a dose regimen beginning with a first cycle comprising an initial dose of between 0.25 μg/kg and 4 μg/kg, and sequential cycles wherein the dose is increased two to three times over the previous dose, and wherein the IL-15/IL-15Rα complex is provided as part of a pharmaceutical composition as described herein, e.g. as a liquid pharmaceutical composition or as a solid pharmaceutical composition as described herein. Each dose is administered at least once, twice, four times or six times before elevating the dose to the next level, and the concentration of free IL-15 in a sample (e.g. a plasma sample) obtained from the subject a certain period of time after the administration of a dose of the IL-15/IL-15Rα complex, e.g. as disclosed herein, (e.g. approximately 24 hours to approximately 48 hours, approximately 24 hours to approximately 36 hours, approximately 24 hours to approximately 72 hours, approximately 48 hours to approximately 72 hours, approximately 36 hours to approximately 48 hours, or approximately 48 hours to 60 hours after the administration of a dose of the IL-15/IL-15Rα complex and before the administration of another dose of the IL-15/IL-15Rα complex is monitored before elevating the dose to the next level, and wherein the IL-15/IL-15Rα complex is provided as part of a pharmaceutical composition as described herein, e.g. as a liquid pharmaceutical composition or as a solid pharmaceutical composition as described herein.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Rα complex, e.g. as disclosed herein, to the subject in a dose regimen at the following sequential doses: (i) 0.25 μg/kg; (ii) 0.5 μg/kg; (iii) 1 μg/kg; (iv) 2 μg/kg; (v) 4 μg/kg; and (vi) 8 μg/kg, and wherein the IL-15/IL-15Rα complex is provided as part of a pharmaceutical composition as described herein, e.g. as a liquid pharmaceutical composition or as a solid pharmaceutical composition as described herein. In a certain embodiment, the IL-15/IL-15Rα complex, e.g. as disclosed herein, is administered to the subject in a dose regimen at the following sequential doses: (i) 1 μg/kg; (ii) 2 μg/kg; (iii) 4 μg/kg; and (iv) 8 μg/kg, and wherein the IL-15/IL-15Rα complex is provided as part of a pharmaceutical composition as described herein, e.g. as a liquid pharmaceutical composition or as a solid pharmaceutical composition as described herein. Each dose is administered at least once, twice, four times or six times in a dosing cycle before elevating the dose to the next level, and wherein the concentration of free IL-15 in a sample (e.g. a plasma sample) obtained from the subject a certain period of time after the administration of a dose of the IL-15/IL-15Rα complex, e.g. as disclosed herein (e.g. approximately 24 hours to approximately 48 hours, approximately 24 hours to approximately 36 hours, approximately 24 hours to approximately 72 hours, approximately 48 hours to approximately 72 hours, approximately 36 hours to approximately 48 hours, or approximately 48 hours to 60 hours after the administration of a dose of the IL-15/IL-15Rα complex, and before the administration of another dose of the IL-15/IL-15Rα complex, is monitored before elevating the dose to the next level, and wherein the IL-15/IL-15Rα complex is provided as part of a pharmaceutical composition as described herein, e.g. as a liquid pharmaceutical composition or as a solid pharmaceutical composition as described herein.

In another embodiment, provided herein is a method for preventing, treating and/or managing cancer in a subject, method comprising administering an IL-15/IL-15Rα complex, e.g. as disclosed herein, to the subject in an dose regimen at the following sequential doses: (i) 1 μg/kg; (ii) 2 μg/kg; (iii) 4 μg/kg; and (iv) 8 μg/kg, wherein each dose is administered at least at least once, twice, four times or six times in a dosing cycle before elevating the dose to the next level, and wherein the IL-15/IL-15Rα complex is provided as part of a pharmaceutical composition as described herein, e.g. as a liquid pharmaceutical composition or as a solid pharmaceutical composition as described herein. In a specific embodiment, the method comprises administering the IL-15/IL-15Rα complex, e.g. as disclosed herein, to the subject using a cyclical administration regimen, wherein the cyclical administration regimen comprises: (a) administering subcutaneously to the subject a dose of 0.1 to 10 μg/kg of the IL-15/IL-15Rα complex every 1, 2 or 3 days over a first period of 1 week to 3 weeks; and (b) after a second period of 1 week to 2 months in which no IL-15/IL-15Rα complex is administered to the subject, administering subcutaneously to the subject a dose of 0.1 to 10 μg/kg of the IL-15/IL-15Rα complex every 1, 2 or 3 days over a third period of 1 week to 3 weeks, and wherein the IL-15/IL-15Rα complex is provided as part of a pharmaceutical composition as described herein, e.g. as a liquid pharmaceutical composition or as a solid pharmaceutical composition as described herein.

In a particular embodiment, the subject is a human subject. In certain embodiments, the dose in the treatment cycle is administered 1, 2, 3, 4, 5, 6, 7, 8, 9, 10 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times. In some embodiments, the dose is administered 1, 2, 3, 4, 5, 6, 7, 8, 9, 10 or more times, or 1 to 3, 1 to 4, 1 to 5, 2 to 4, 2 to 5, 2 to 6, 1 to 6, 3 to 6, 4 to 6 or 6 to 8 times over a 5 to 7 day, 5 to 10 day, 7 to 12 day, 7 to 14 day, 7 to 21 day or 14 to 21 day period of time. In certain embodiments, each dose is administered 1, 2, 3, 4, 5, 6, 7, 8, 9, 10 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times, per dosing cycle. In specific embodiments, each dose is administered at least 1, 2, 3, 4, 5, 6 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times over a 5 to 7 day, 5 to 10 day, 7 to 12 day, 7 to 14 day, 7 to 21 day or 14 to 21 day period of time.

In another specific embodiment, the subject is administered a dose three times per 7 day week (e.g. Monday, Wednesday and Friday). In certain embodiments, the subject is monitored for the following adverse events, such as grade 3 or 4 thrombocytopenia, grade 3 or 4 granulocytopenia, grade 3 or 4 leukocytosis (White Blood Cell (WBC)>100,000 mm3), grade 3 or 4 decreases in WBC, absolute lymphocyte count (ALC) and/or absolute neutrophil count (ANC), lymphocytosis, and organ dysfunction (e.g. liver or kidney dysfunction). In certain embodiments, the dose is not increased and the dose may be remain the same, be stopped or reduced if the subject experiences adverse events, such as grade 3 or 4 thrombocytopenia, grade 3 or 4 granulocytopenia, grade 3 or leukocytosis (White Blood Cell>100,000 mm3), grade 3 or 4 decreases in WBC, absolute lymphocyte count (ALC) and/or absolute neutrophil count (ANC), lymphocytosis, and organ dysfunction (e.g. liver or kidney dysfunction). In accordance with these embodiments, the dose of the IL-15/IL-15Rα complex, e.g. as disclosed herein, administered to the subject may be reduced or remain the same until the adverse events decrease or disappear.

In specific embodiments, in accordance with the methods described herein, each dose is administered once a week for three weeks. In specific embodiments, in accordance with the methods described herein, each dose is administered once, three times a week for two weeks. In specific embodiments, in accordance with the methods described herein, each dose is administered once, three times a week for two, three, or four weeks. In specific embodiments, in accordance with the methods described herein, each dose is administered once, six times a week for two, three, or four weeks. In specific embodiments, in accordance with the methods described herein, each dose is administered once, every other day, for two, three, or four weeks. In specific embodiments, in accordance with the methods described herein, each dose is administered once, every day, for two, three, or four weeks.

In certain embodiments, the IL-15/IL-15Rα complex, e.g. as disclosed herein, is administered subcutaneously to a subject in accordance with the methods described herein, and wherein the IL-15/IL-15Rα complex is provided as part of a pharmaceutical composition as described herein, e.g. as a liquid pharmaceutical composition or as a solid pharmaceutical composition as described herein. In some embodiments, the IL-15/IL-15Rα complex, e.g. as disclosed herein, is administered intravenously or intramuscularly to a subject in accordance with the methods described herein, and wherein the IL-15/IL-15Rα complex is provided as part of a pharmaceutical composition as described herein, e.g. as a liquid pharmaceutical composition or as a solid pharmaceutical composition as described herein. In certain embodiments, the IL-15/IL-15Rα complex, e.g. as disclosed herein, is administered intratumorally to a subject in accordance with the methods described herein, and wherein the IL-15/IL-15Rα complex is provided as part of a pharmaceutical composition as described herein, e.g. as a liquid pharmaceutical composition or as a solid pharmaceutical composition as described herein. In some embodiments, the IL-15/IL-15Rα complex, e.g. as disclosed herein, is administered locally to a site (e.g. a site of infection) in a subject in accordance with the methods described herein, and wherein the IL-15/IL-15Rα complex is provided as part of a pharmaceutical composition as described herein, e.g. as a liquid pharmaceutical composition or as a solid pharmaceutical composition as described herein.

In certain embodiments, a sample obtained from a subject in accordance with the methods described herein is a blood sample. In a specific embodiment, the sample is a plasma sample. Basal plasma levels of IL-15 are approximately 1 pg/ml in humans, approximately 8-10 μg/ml in monkeys (such as macaques), and approximately 12 pg/ml in rodents (such as mice). Techniques known to one skilled in the art can be used to obtain a sample from a subject.

The plasma levels of IL-15 can be assessed using standard techniques known to one of skill in the art. For example, plasma can be obtained from a blood sample obtained from a subject and the levels of IL-15 in the plasma can be measured by ELISA.

In specific embodiments, examples of immune function enhanced by the methods described herein include the proliferation/expansion of lymphocytes (e.g. increase in the number of lymphocytes), inhibition of apoptosis of lymphocytes, activation of dendritic cells (or antigen presenting cells), and antigen presentation. In particular embodiments, an immune function enhanced by the methods described herein is proliferation/expansion in the number of or activation of CD4⁺ T cells (e.g. Th1 and Th2 helper T cells), CD8⁺ T cells (e.g. cytotoxic T lymphocytes, alpha/beta T cells, and gamma/delta T cells), B cells (e.g. plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells). In one embodiment, the methods described herein enhance the proliferation/expansion or number of lymphocyte progenitors. In some embodiments, the methods described herein increases the number of CD4⁺ T cells (e.g. Th1 and Th2 helper T cells), CD8⁺ T cells (e.g. cytotoxic T lymphocytes, alpha/beta T cells, and gamma/delta T cells), B cells (e.g. plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells) by approximately 1 fold, 2 fold, 3 fold, 4 fold, 5 fold, 6 fold, 7 fold, 8 fold, 9 fold, 10 fold, 20 fold, or more relative to a negative control.

In a specific embodiment, the methods described herein enhance or induce immune function in a subject by at least 0.2 fold, 0.5 fold, 0.75 fold, 1 fold, 1.5 fold, 2 fold, 2.5 fold, 3 fold, 4 fold, 5 fold, 6 fold, 7 fold, 8 fold 9 fold, or at least 10 fold relative to the immune function in a subject not administered the combination of an IL-15/IL-15Rα complex, e.g. as disclosed herein, and an anti-PD-1 antibody molecule using assays well known in the art, e.g. ELISPOT, ELISA, and cell proliferation assays, and wherein the IL-15/IL-15Rα complex is provided as part of a pharmaceutical composition as described herein, e.g. as a liquid pharmaceutical composition or as a solid pharmaceutical composition as described herein. In a specific embodiment, the methods described herein enhance or induce immune function in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the immune function in a subject not administered the combination of an IL-15/IL-15Rα complex, e.g. as disclosed herein, and an anti-PD-1 antibody molecule using assays well known in the art, e.g. ELISPOT, ELISA, and cell proliferation assays, and wherein the IL-15/IL-15Rα complex is provided as part of a pharmaceutical composition as described herein, e.g. as a liquid pharmaceutical composition or as a solid pharmaceutical composition as described herein. In a specific embodiment, the immune function is cytokine release (e.g. interferon-gamma, IL-2, IL-5, IL-10, IL-12, or transforming growth factor (TGF)-beta). In one embodiment, the IL-15 mediated immune function is NK cell proliferation, which can be assayed, e.g. by flow cytometry to detect the number of cells expressing markers of NK cells (e.g. CD56). In one embodiment, the IL-15 mediated immune function is CD8+ T cell proliferation, which can be assayed, e.g. by flow. In another embodiment, the IL-15 mediated immune function is antibody production, which can be assayed, e.g. by ELISA. In some embodiments, the IL-15 mediated immune function is effector function, which can be assayed, e.g. by a cytotoxicity assay or other assays well known in the art. The effect of one or more doses of a combination of an IL-15/IL-15Rα complex and an anti-PD-1 antibody molecule on peripheral blood lymphocyte counts can be monitored/assessed using standard techniques known to one of skill in the art. Peripheral blood lymphocytes counts in a mammal can be determined by, e.g. obtaining a sample of peripheral blood from said mammal, separating the lymphocytes from other components of peripheral blood such as plasma using, e.g. FicollHypaque (Pharmacia) gradient centrifugation, and counting the lymphocytes using trypan blue. Peripheral blood T-cell counts in mammal can be determined by, e.g. separating the lymphocytes from other components of peripheral blood such as plasma using, e.g. a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS. Further, the effect on a particular subset of T cells (e.g. CD2⁺, CD4⁺, CD8⁺, CD4⁺RO⁺, CD8⁺RO⁺, CD4⁺RA⁺, or CD8⁺RA⁺) or NK cells can be determined using standard techniques known to one of skill in the art such as FACS.

### Combination Therapy

Other therapies that can be used in combination with the IL-15/IL-15Rα complex, e.g. as disclosed herein, are also provided. In one aspect, provided herein are methods for preventing, treating, and/or managing cancer, comprising administering an effective amount of an IL-15/IL-15Rα complex, e.g. as disclosed herein, and at least one additional therapeutic agent, wherein the IL-15/IL-15Rα complex is provided as part of a pharmaceutical composition as described herein, e.g. as a liquid pharmaceutical composition or as a solid pharmaceutical composition as described herein.

In one embodiment the at least one additional therapeutic agent is an anti-PD-1 antibody.

In a preferred embodiment, the anti-PD-1 antibody is pembrolizumab, nivolumab, cemiplimab, spartalizumab, camrelizumab, sintilimab, tislelizumab or toripalimab.

In a particularly preferred embodiment, the anti-PD-1 antibody is spartalizumab.

In specific embodiments, the administration of a combination of an IL-15/IL-15Rα complex, e.g. as disclosed herein, and an anti-PD-1 antibody molecule to a subject in accordance with the methods described herein achieves one, two, or three or more results: (1) a reduction in the growth of a tumor or neoplasm; (2) a reduction in the formation of a tumor; (3) an eradication, removal, or control of primary, regional and/or metastatic cancer; (4) a reduction in metastatic spread; (5) a reduction in mortality; (6) an increase in survival rate; (7) an increase in length of survival; (8) an increase in the number of patients in remission; (9) a decrease in hospitalization rate; (10) a decrease in hospitalization lengths; and (11) the maintenance in the size of the tumor so that it does not increase by more than 10%, or by more than 8%, or by more than 6%, or by more than 4%, or by more than 2%, wherein the IL-15/IL-15Rα complex is provided as part of a pharmaceutical composition as described herein, e.g. as a liquid pharmaceutical composition or as a solid pharmaceutical composition as described herein.

In a specific embodiment, the administration of a combination of an IL-15/IL-15Rα complex, e.g. as disclosed herein, and an anti-PD-1 antibody molecule to a subject with cancer in accordance with the methods described herein inhibits or reduces the growth of a tumor by at least 2 fold, preferably at least 2.5 fold, at least 3 fold, at least 4 fold, at least 5 fold, at least 7 fold, or at least 10 fold relative to the growth of a tumor in a subject with cancer administered a negative control as measured using assays known in the art, and wherein the IL-15/IL-15Rα complex is provided as part of a pharmaceutical composition as described herein, e.g. as a liquid pharmaceutical composition or as a solid pharmaceutical composition as described herein. In another embodiment, the administration of a combination of an IL-15/IL-15Rα complex, e.g. as disclosed herein, and an anti-PD-1 antibody molecule to a subject with cancer in accordance with the methods described herein inhibits or reduces the growth of a tumor by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to the growth of a tumor in a subject with cancer administered a negative control, or an IL-15/IL-15Rα complex, e.g. as disclosed herein, or an anti-PD-1 antibody molecule as a single agent, as measured using assays known in the art, and wherein the IL-15/IL-15Rα complex is provided as part of a pharmaceutical composition as described herein, e.g. as a liquid pharmaceutical composition or as a solid pharmaceutical composition as described herein.

Examples of cancerous disorders include, but are not limited to, solid tumors, hematological cancers, soft tissue tumors, and metastatic lesions. Examples of solid tumors include malignancies, e.g. sarcomas, and carcinomas (including adenocarcinomas and squamous cell carcinomas), of the various organ systems, such as those affecting liver, lung, breast, lymphoid, gastrointestinal (e.g. colon), genitourinary tract (e.g. renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g. in the lung, esophagus, skin, head and neck region, oral cavity, anus, and cervix. In one embodiment, the cancer is a melanoma, e.g. an advanced stage melanoma. Metastatic lesions of the aforementioned cancers can also be treated or prevented using the methods and compositions of the invention.

Exemplary cancers whose growth can be inhibited using the combination an IL-15/IL-15Rα complex, e.g. as disclosed herein, and an anti-PD-1 antibody molecule include cancers typically responsive to immunotherapy. Non-limiting examples of preferred cancers for treatment include melanoma (e.g. metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer). Additionally, refractory or recurrent malignancies can be treated using the combination therapy described herein.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodysplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g. mesothelioma), and combinations of said cancers.

In a specific embodiment, the cancer is melanoma, renal cancer, colon cancer, or prostate cancer. In one embodiment, the cancer is melanoma. In another embodiment, the cancer is metastatic. in another embodiment the cancer is metastatic melanoma. In another embodiments, the subject has been previously treated with immune checkpoint inhibitor (CPI), for example, anti-PD-1 and/or anti-PD-L1, and/or anti CTLA-4, and has responded and progressed.

The combination of IL-15/IL-15Rα complex, e.g. as disclosed herein, and anti-PD-1 antibody molecule can be administered together with one or more other therapies, e.g. anti-cancer agents, cytokines or anti-hormonal agents, to treat and/or manage cancer, wherein the IL-15/IL-15Rα complex is provided as part of a pharmaceutical composition as described herein, e.g. as a liquid pharmaceutical composition or as a solid pharmaceutical composition as described herein. Non-limiting exemplary anti-cancer agents are described below.

In one embodiment, provided herein is a method for preventing, treating and/or managing disorders in a subject, e.g. a hyperproliferative condition or disorder (e.g. a cancer) in a subject comprising administering an IL-15/IL-15Rα complex, e.g. as disclosed herein, and an anti-PD-1 antibody molecule to a subject in need thereof, and wherein the IL-15/IL-15Rα complex is provided as part of a pharmaceutical composition as described herein, e.g. as a liquid pharmaceutical composition or as a solid pharmaceutical composition as described herein. In some embodiments, the anti-PD-1 antibody molecule is administered by injection (e.g. subcutaneously or intravenously) at a dose (e.g. a flat dose) of about 200 mg to 500 mg, e.g. about 250 mg to 450 mg, about 300 mg to 400 mg, about 250 mg to 350 mg, about 350 mg to 450 mg, or about 300 mg or about 400 mg. The dosing schedule (e.g. flat dosing schedule) can vary from e.g. about once a week to about every 2 weeks, about every 3 weeks, about every 4 weeks, about every 5 weeks, or about every 6 weeks. In one embodiment, the anti-PD-1 antibody molecule is administered at a dose from about 300 mg to 400 mg once about every three weeks or once about every four weeks. In one embodiment, the anti-PD-1 antibody molecule is administered at a dose from about 300 mg once about every three weeks. In one embodiment, the anti-PD-1 antibody molecule is administered at a dose from about 400 mg once about every four weeks. In one embodiment, the anti-PD-1 antibody molecule is administered at a dose from about 300 mg once about every four weeks. In one embodiment, the anti-PD-1 antibody molecule is administered at a dose from about 400 mg once about every three weeks.

In accordance with the methods described herein, the IL-15/IL-15Rα complex, e.g. as disclosed herein, can be administered to a subject in a pharmaceutical composition, e.g. in a liquid pharmaceutical composition as disclosed herein or as a solid pharmaceutical composition as disclosed herein. In one embodiment, the IL-15/IL-15Rα complex, e.g. as disclosed herein, is administered to a subject in a liquid pharmaceutical composition. In another embodiment, the IL-15/IL-15Rα complex, e.g. as disclosed herein, is administered to a subject in a solid pharmaceutical composition. In specific embodiments, the IL-15/IL-15Rα complex, e.g. as disclosed herein, is administered in combination with one or more other therapies, e.g. an anti-PD-1 antibody molecule, wherein the IL-15/IL-15Rα complex is provided as part of a pharmaceutical composition as described herein, e.g. as a liquid pharmaceutical composition or as a solid pharmaceutical composition as described herein. Combination therapy includes concurrent and successive administration of an IL-15/IL-15Rα complex, e.g. as disclosed herein, and an anti-PD-1 antibody molecule, wherein the IL-15/IL-15Rα complex is provided as part of a pharmaceutical composition as described herein, e.g. as a liquid pharmaceutical composition or as a solid pharmaceutical composition as described herein. As used herein, the IL-15/IL-15Rα complex, e.g. as disclosed herein, and the anti-PD-1 antibody molecule are said to be administered concurrently if they are administered to the patient on the same day, for example, simultaneously, or about 1, about 2, about 3, about 4, about 5, about 6, about 7, or about 8 hours apart. In contrast, the IL-15/IL-15Rα complex, e.g. as disclosed herein, and the anti-PD-1 antibody molecule are said to be administered successively if they are administered to the patient on different days, for example, the IL-15/IL-15Rα complex, e.g. as disclosed herein, and the anti-PD-1 antibody molecule can be administered at a 1-day, 2-day or 3-day interval. In the methods and uses described herein, administration of the IL-15/IL-15Rα complex, e.g. as disclosed herein, can precede or follow administration of the anti-PD-1 antibody molecule. When administered simultaneously, the IL-15/IL-15Rα complex, e.g. as disclosed herein, and the anti-PD-1 antibody molecule can be in the same pharmaceutical composition or in a different pharmaceutical composition.

The combination of an IL-15/IL-15Rα complex, e.g. as disclosed herein, and an anti-PD-1 antibody molecule can also be administered together with radiation therapy comprising, e.g. the use of x-rays, gamma rays and other sources of radiation to destroy the cancer cells. In specific embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass. The IL-15/IL-15Rα complex, e.g. as disclosed herein, and anti-PD-1 antibody molecule can also be administered in combination with chemotherapy. In one embodiment, the IL-15/IL-15Rα complex, e.g. as disclosed herein, and anti-PD-1 antibody molecule can be administered in accordance with the methods or uses described herein before, during or after radiation therapy or chemotherapy. In one embodiment, a combination of the IL-15/IL-15Rα complex, e.g. as disclosed herein, and anti-PD-1 antibody molecule can be administered before, during or after surgery.

In some embodiments, the combination of the IL-15/IL-15Rα complex, e.g. as disclosed herein, and anti-PD-1 antibody molecule is administered to a subject suffering from or diagnosed with cancer, wherein the IL-15/IL-15Rα complex is provided as part of a pharmaceutical composition as described herein, e.g. as a liquid pharmaceutical composition or as a solid pharmaceutical composition as described herein. In other embodiments, the combination of the IL-15/IL-15Rα complex, e.g. as disclosed herein, and anti-PD-1 antibody molecule is administered to a subject predisposed or susceptible to developing cancer, wherein the IL-15/IL-15Rα complex is provided as part of a pharmaceutical composition as described herein, e.g. as a liquid pharmaceutical composition or as a solid pharmaceutical composition as described herein.

In certain embodiments, the combination of the IL-15/IL-15Rα complex, e.g. as disclosed herein, and anti-PD-1 antibody molecule is administered to a subject which is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In other embodiments, the combination of the IL-15/IL-15Rα complex, e.g. as disclosed herein, and anti-PD-1 antibody molecule is administered to a human adult. In certain embodiments, the combination of the IL-15/IL-15Rα complex, e.g. as disclosed herein, and anti-PD-1 antibody molecule is administered to a subject that is, will or has undergone surgery, chemotherapy and/or radiation therapy. In some embodiments, the combination of the IL-15/IL-15Rα complex, e.g. as disclosed herein, and anti-PD-1 antibody molecule is administered to refractory patients. In one embodiment, refractory patient is a patient refractory to a standard anti-cancer therapy. In one embodiment, a patient with cancer, is refractory to a therapy when the cancer has not significantly been eradicated and/or the symptoms have not been significantly alleviated. The determination of whether a patient is refractory can be made either in vivo or in vitro by any method known in the art for assaying the effectiveness of a treatment, using art-accepted meanings of “refractory” in such a context. In various embodiments, a patient with cancer is refractory when a cancerous tumor has not decreased or has increased.

Other methods and uses contemplated herein used to treat patients that have been exposed to particular toxins or pathogens. Accordingly, in another aspect provided is a method of treating an infectious disease in a subject comprising administering to the subject a combination as disclosed herein, e.g. a combination including the IL-15/IL-15Rα complex, e.g. as disclosed herein, and an anti-PD-1 antibody molecule, such that the subject is treated for the infectious disease.

In the treatment of infection (e.g. acute and/or chronic), administration of the combination of the IL-15/IL-15Rα complex, e.g. as disclosed herein, and anti-PD-1 antibody molecule can be combined with conventional treatments in addition to or in lieu of stimulating natural host immune defenses to infection. Natural host immune defenses to infection include, but are not limited to inflammation, fever, antibody-mediated host defense, T-lymphocyte-mediated host defenses, including lymphokine secretion and cytotoxic T-cells (especially during viral infection), complement mediated lysis and opsonization (facilitated phagocytosis), and phagocytosis. The ability of the anti-PD-1 antibody molecules to reactivate dysfunctional T-cells is useful to treat chronic infections, in particular those in which cell-mediated immunity is important for complete recovery.

Antibody mediated PD-1 blockade can act as an adjuvant to IL-15/IL-15Rα complex administration or in combination with an IL-15/IL-15Rα complexes and/or vaccines, to stimulate the immune response to pathogens, toxins and self-antigens. Examples of pathogens for which this therapeutic approach is particularly useful include pathogens for which there is currently no effective vaccine, or pathogens for which conventional vaccines are less than completely effective. These include, but are not limited to human immunodeficiency virus (HIV), hepatitis virus (A, B and/or C), influenza virus, herpes simplex virus, giardia, Plasmodium species, Leishmania, Staphylococcus aureus, Pseudomonas aeruginosa. Immune system stimulation by IL-15/IL-15Rα complexes and PD-1 blockade is particularly useful against established infections by agents such as HIV that present altered antigens over the course of the infections. These novel epitopes are recognized as foreign at the time of treatment, thus provoking a strong T cell response that is not dampened by negative signals through PD-1, for example.

Other therapies that can be used in combination with the IL-15/IL-15Rα complex, e.g. as disclosed herein, and anti-PD-1 antibody molecule, for the prevention, treatment and/or management of a disease, e.g. cancer, infectious disease, lymphopenia, immunodeficiency and wounds, include, but are not limited to, small molecules, synthetic drugs, peptides (including cyclic peptides), polypeptides, proteins, nucleic acids (e.g. DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such therapies include, but are not limited to, immunomodulatory agents (e.g. interferon), anti-inflammatory agents (e.g. adrenocorticoids, corticosteroids (e.g. beclomethasone, budesonide, flunisolide, fluticasone, triamcinolone, methylprednisolone, prednisolone, prednisone, hydrocortisone), glucocorticoids, steroids, and non-steroidal anti-inflammatory drugs (e.g. aspirin, ibuprofen, diclofenac, and COX-2 inhibitors), pain relievers, leukotriene antagonists (e.g. montelukast, methyl xanthines, zafirlukast, and zileuton), beta2-agonists (e.g. albuterol, biterol, fenoterol, isoetharie, metaproterenol, pirbuterol, salbutamol, terbutalin formoterol, salmeterol, and salbutamol terbutaline), anticholinergic agents (e.g. ipratropium bromide and oxitropium bromide), sulphasalazine, penicillamine, dapsone, antihistamines, anti-malarial agents (e.g. hydroxychloroquine), anti-viral agents (e.g. nucleoside analogs (e.g. zidovudine, acyclovir, ganciclovir, vidarabine, idoxuridine, trifluridine, and ribavirin), foscarnet, amantadine, rimantadine, saquinavir, indinavir, ritonavir, and AZT) and antibiotics (e.g. dactinomycin (formerly actinomycin), bleomycin, erythromycin, penicillin, mithramycin, and anthramycin).

Any therapy which is known to be useful, or which has been used or is currently being used for the prevention, management, and/or treatment of a disease that is affected by IL-15 function/signaling and/or immune-checkpoint modulation can be used in combination with a combination therapy of an IL-15/IL-15Rα complex, e.g. as disclosed herein, and anti-PD-1 antibody molecule. See, e.g. Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996, and Physicians' Desk Reference (66th ed. 2012) for information regarding therapies (e.g. prophylactic or therapeutic agents) which have been or are currently being used for preventing, treating and/or managing disease or disorder, e.g. cancer, infectious disease, lymphopenia, immunodeficiency and wounds.

Non-limiting examples of one or more other therapies that can be used in addition to a combination therapy of an IL-15/IL-15Rα complex, e.g. as disclosed herein, and anti-PD-1 antibody molecule include immunomodulatory agents, such as but not limited to, chemotherapeutic agents and non-chemotherapeutic immunomodulatory agents. Non-limiting examples of chemotherapeutic agents include methotrexate, cyclosporin A, leflunomide, cisplatin, ifosfamide, taxanes such as taxol and paclitaxol, topoisomerase 1 inhibitors (e.g. CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracene dione, mitoxantrone, mithramycin, actinomycin D, 1-dehydrotestosterone, glucocorticoids, procaine, tetracaine, lidocaine, propranolol, and puromycin homologs, and cyclophosphamide. Biological Activity

The IL-15/IL-15Rα complex, e.g. as disclosed herein, and/or anti-PD-1 antibody molecule increases an immune response that can be, e.g. an antibody response (humoral response) or a cellular immune response, e.g. cytokine secretion (e.g. interferon-gamma), helper activity or cellular cytotoxicity. In one embodiment, the increased immune response is increased cytokine secretion, antibody production, effector function, T cell proliferation, and/or NK cell proliferation. Various assays to measure such activities are well known in the art, and include enzyme-linked immunosorbent assays (ELISA; see e.g. in Section 2.1 of Current Protocols in Immunology, Coligan et al. (eds.), John Wiley and Sons, Inc. 1997), a “tetramer staining” assay to identify antigen-specific T-cells (see Altman et al., (1996), Science 274: 94-96), a mixed lymphocyte target culture assay (see e.g. in Palladino et al., (1987), Cancer Res. 47:5074-5079) and an ELISPOT assay that can be used to measure cytokine release in vitro (see, e.g. Scheibenbogen et al., (1997), Int. J. Cancer 71:932-936).

The immune response induced or enhanced by a combination of IL-15/IL-15Rα complex, e.g. as disclosed herein, and anti-PD-1 antibody molecule is enhanced or increased by at least 2 fold, 3 fold, 4 fold, 5 fold, 6 fold, 7 fold, 8 fold, 9 fold, 10 fold, 11 fold, or 12 fold relative to an immune response elicited by a negative control, or by an IL-15/IL-15Rα complex, e.g. as disclosed herein, or an anti-PD-1 antibody molecule administered as a single agent, as assayed by any known method in the art. In certain embodiments, the immune response induced by the combination of an IL-15/IL-15Rα complex, e.g. as disclosed herein, and an anti-PD-1 antibody molecule is enhanced by at least 0.5-2 times, at least 2-5 times, at least 5-10 times, at least 10-50 times, at least 50-100 times, at least 100-200 times, at least 200-300 times, at least 300-400 times or at least 400-500 times relative to the immune response induced by a negative control as assayed by any known method in the art. In some embodiments, the assay used to assess immune response measures the level of antibody production, cytokine production, or cellular cytotoxicity. In some embodiments, the assay used to measure the immune response is an enzyme-linked immunosorbent assay (ELISA) that determines antibody or cytokine levels, an ELISPOT assay that determines cytokine release, or a [⁵¹Cr] release assay that determines cellular cytotoxicity.

In a specific embodiment, the combination of an IL-15/IL-15Rα complex, e.g. as disclosed herein, and an anti-PD-1 antibody molecule increases the expression of IL-2 on whole blood activated by Staphylococcal enterotoxin B (SEB). For example, IL-15/IL-15Rα complex, e.g. as disclosed herein, and anti-PD-1 antibody molecule increases the expression of IL-2 by at least about 2 fold, about 3 fold, about 4 fold, or about 5 fold, compared to the expression of IL-2 when an the IL-15/IL-15Rα complex, e.g. as disclosed herein, the anti-PD-1 antibody molecule or an isotype control (e.g. IgG4) is used alone.

In one embodiment, the proliferation or viability of cancer cells contacted with a combination of an IL-15/IL-15Rα complex, e.g. as disclosed herein, and an anti-PD-1 antibody molecule is inhibited or reduced by at least about 2 fold, preferably at least about 2.5 fold, at least about 3 fold, at least about 4 fold, at least about 5 fold, at least about 7 fold, or at least about 10 fold relative to the proliferation of the cancer cells when contacted with a negative control or an IL-15/IL-15Rα complex, e.g. as disclosed herein, or an anti-PD-1 antibody molecule as a single agent, as measured using assays known in the art, e.g. cell proliferation assays using CSFE, BrdU, or radioactive thymidine incorporation. Alternatively, cell viability can be measured by assays that measure lactate dehydrogenase (LDH), a stable cytosolic enzyme that is released upon cell lysis, or by the release of [⁵¹Cr] upon cell lysis. In another embodiment, the proliferation of cancer cells contacted with a combination of an IL-15/IL-15Rα complex, e.g. as disclosed herein, and an anti-PD-1 antibody molecule is inhibited or reduced by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to cancer cells contacted with a negative control or an IL-15/IL-15Rα complex or an anti-PD-1 antibody molecule as a single agent, as measured using assays known in the art, e.g. cell proliferation assays using CSFE, BrdU, or radioactive thymidine incorporation.

Cancer cell lines on which such assays can be performed are known to those of skill in the art. Necrosis, apoptosis and proliferation assays can also be performed on primary cells, e.g. a tissue explant.

The details of one or more embodiments of the disclosure are set forth in the accompanying description above. Furthermore, it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments. Although any methods and materials similar or equivalent to those described herein can be used in the practice or testing of the present disclosure, the preferred methods and materials are now described. Other features, objects, and advantages of the disclosure will be apparent from the description and from the claims.

### MODES FOR CARRYING OUT THE INVENTION

The following Examples are presented in order to more fully illustrate the preferred embodiments of the disclosure. One of ordinary skill in the art will recognize the numerous modifications and variations that may be performed without altering the spirit or scope of the disclosure. Such modifications and variations are encompassed within the scope of the disclosure. These examples should in no way be construed as limiting the scope of the disclosed matter, as defined by the appended claims.

### EXAMPLE 1

The aim of this study was assess the stability of a liquid pharmaceutical composition comprising heterodimeric IL-15/IL-15Rα complex with IL-15 comprising SEQ ID NO:2 and IL-15Rα comprising SEQ ID NO:5 in a head to head comparison of three different formulations for long storage at 2-8° C. Additionally, stability under stressed (40° C.) and accelerated (25° C.) conditions was assessed as well as freeze-thaw and shaking stress was applied to compositions filled in 1.2 mL vials

### Materials and Equipment

Heterodimeric IL-15/IL-15Rα complex with SEQ ID NO:2 and SEQ ID NO:5, respectively, was provided at a concentration of 10 mg/mL in 5 mM histidine at pH 6.5. A total of 3 formulations were evaluated in the course of this study as detailed in Table 2.

TABLE 2
Target composition

| | complex | | | Buffer | | Stabilizer | | |
|---|---|---|---|---|---|---|---|---|
| Formulation | conc. | | | Conc. | | conc. | | Surfactant |
| ID | (mg/mL) | pH | Buffer | (mM) | Stabilizer | (mM) | Surfactant | concentration |
| F1 | 1 | 5.5 | Na-Acetate | 20 | Sucrose | 260 | Polysorbate 20 | 0.04% |
| F2 | 1 | 5.5 | Histidine | 20 | Sucrose | 260 | Polysorbate 20 | 0.04% |
| F3 | 1 | 5.0 | Na-Acetate | 20 | Sucrose | 260 | Poloxamer 188 | 0.2% |

### Preparation of Heterodimeric IL-15/IL-15Rα Complex

The heterodimeric IL-15/IL-15Rα complex in 5 mM histidine at pH 6.5 was thawed in a water bath at 30° C.±5° C. until thawed completely.

In order to achieve acetate-based formulations, the heterodimeric IL-15/IL-15Rα complex was subjected to a buffer exchange: The complex was diluted to 120 ml at a concentration of 1 mg/mL using stock solution E6 (20 mM acetate buffer, pH 5.0) and then split into to a total of 8 spin columns (MWCO 10 kDa) of 15 ml each. The spin columns were centrifuged at 4500 rpm and 10° C. for 10 min. The filtrate was removed, the retentate filled to 15 ml and resuspended and the process repeated for a total of 6 buffer exchange cycles. After the final cycle of centrifugation, filtrate removal, refilling, and resuspension, a concentration step was initiated. Spin columns were centrifuged at 4500 rpm for 10 min. After each centrifugation, the removed volume was replaced with protein solution from the other spin columns, thereby reducing the number of spin columns from 8 to 1 column with the target volume of concentrated drug substance. After concentration of the complex, the concentrated material was transferred to a Nalgene bottle and the concentration determined by Nanodrop. Concentrated solutions were diluted to a concentration of 10±0.5 mg/mL with their respective buffers to simplify handling during compounding.

### Compounding and Filtration

Histidine-based formulation were compounded by adding appropriate stock solutions to achieve the final composition detailed in Table 2. Compounding was performed directly in Nalgene bottles with a minimum volume of 60 ml. The following amounts of stock solutions were added for the different formulations as appropriate:

$${V}_{Complex , Acetate} = \frac{55 ml * 1 mg \text{/} ml}{10 mg / ml} = 5.5 ml$$

$${V}_{Complex in His} = \frac{55 ml * 1 mg \text{/} ml}{10.95 mg / ml} = 5.02 ml$$

$${V}_{Sucrose} = \frac{55 ml * 260 mM}{2000 mM} = 7.15 ml$$

$${V}_{PS 20} = \frac{55 ml * 0.04 \%}{2 \%} = 1.1 ml$$

$${V}_{Poloxamer} = \frac{55 ml * 0.2 \%}{5 \%} = 2.2 ml$$

$${V}_{buffer ( Acetate )} = \frac{( 55 - 5.5 ) ml * 20 mM}{500 mM} = 1.98 ml$$

$${V}_{buffer ( His )} = \frac{55 ml * 20 mM - 5.02 ml * 5 mM}{500 mM} = 2.15 ml$$

Volume was then adjusted to 50 ml and the pH determined and adjusted using 1 M NaOH (acetate buffer) and 1 M HCl (histidine buffer). Changes in density were disregarded. The volume added was recorded, and the solution finally filled up to 55 ml with MilliQ water. Formulations were sterile-filtered through a 0.22 μm PVDF filter under a Laminar Flow and aliquoted in 6R vials each filled with 1.2 mL. The vials were crimped, labelled accordingly and stored according to the stability plan described below. At the respective pullpoint, all samples are analyzed according to the analytical plan outlined below.

### Stability Study and Analysis

The samples were subjected to a stability study with the timepoints and storage conditions as described in Table 3 and analyzed as detailed in

Table 4.

TABLE 3
Storage/stress conditions, analytical time points
for heterodimeric IL-15/IL-15Rα complex

| Time point | | T = 0 | T = 6 W | T = 3M | T = 6M | T = 12M |
|---|---|---|---|---|---|---|
| 2-8° | C. | X | X | X | X | X |
| 25° | C. | | X | X | — | — |
| 40° | C. | | X | — | — | — |

TABLE 4
Applied Test Method, Instrument, Sample Preparation

| Test | Instrument | Sample Preparation |
|---|---|---|
| pH | Metrohm 691 | Not required |
| Turbidity | Hach Lange 2100AN | Not Required |
| Subvisible particles by | PAMAS SVSS | Not Required |
| PAMAS | | |
| Purity by SEC | Suitable HPLC system, e.g | Dilution with mobile phase |
| | Agilent LC1100/1200 | to 0.5 mg/mL |
| Purity by CE-SDS | CE system Beckman PA800 | Dilution with water to 0.75 |
| | Plus or enhanced | mg/mL |
| Purity by RP-HPLC | Suitable HPLC system, e.g | Dilution with water to 0.5 |
| | Agilent LC1100/1200 | mg/mL |
| Charged variants by AEX | Suitable HPLC system, e.g | Dilution to 0.8 mg/mL in |
| | Agilent LC1100/1200 | water and deglycosylation |
| | | enzymes |

### Purity by SEC

This test is based on size exclusion chromatography (SEC) with UV detection. Variants of heterodimeric IL-15/IL-15Rα complex of different size (e.g. lower and higher molecular weight variants and related substances) are separated by SEC under native conditions on a suitable column. The purity of the main peak as well as the amounts of aggregates and fragments was determined as a percentage of the total area obtained for the sample in each chromatogram.

### Purity by CE-SDS

Capillary Electrophoresis SDS (CE-SDS) separates proteins according to their size in an electric field by adding a hydrophilic sieving polymer to the separation buffer. The samples were injected at the inlet side of the capillary and the separation was performed on the long part of the capillary from inlet to the detector. Detection was performed by UV.

### Purity by RP-HPLC

Heterodimeric IL-15/IL-15Rα complex product related substances were separated by Reversed Phase HPLC (RP-HPLC). Depending on the hydrophobicity proteins can be eluted separately from a hydrophobic matrix by applying distinct organic solvent concentrations. A gradient with an increasing amount of acetonitrile was used for the separation on a C8 column. The protein elution was monitored by UV absorption at a wavelength of 215 nm.

### Charge Variants by AEX

Anion Exchange Chromatography (AEX) was used to isolate charge based variants of heterodimeric IL-15/IL-15Rα complex after removal of N and O linked glycans by enzymatic digestion. During the chromatographic separation proteins with an overall negative charge were retained by positively charged functional groups on the stationary phase. By applying a gradient with increasing salt concentration, the weakly bound variants (with a positive charge) eluted first, followed by more and more negatively charged variants. The elution was monitored by UV absorption at 210 nm.

### Results

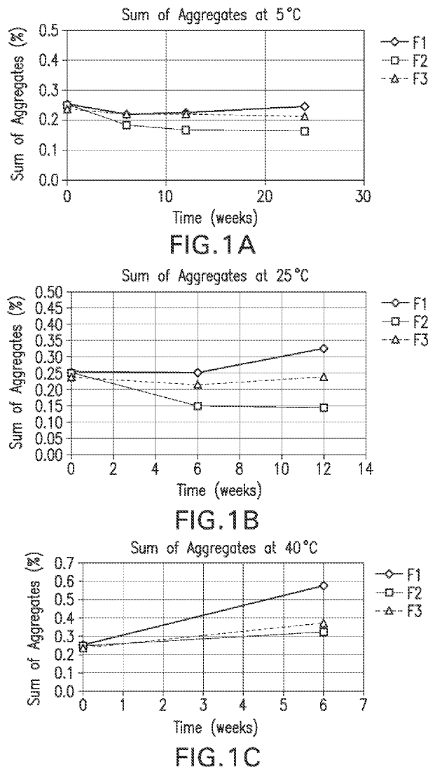

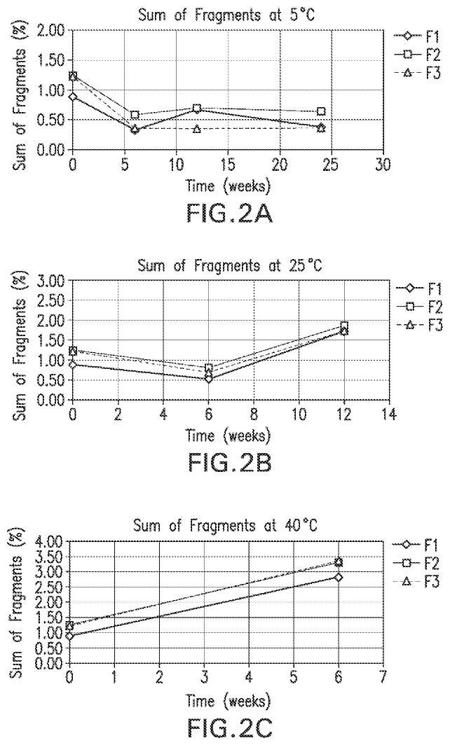

All formulations showed good stability and no significant difference was observed between the tested formulations for the sum of aggregates (see FIG. 1), sum of degradation products by SEC (see FIG. 2), for change of charge variants (see

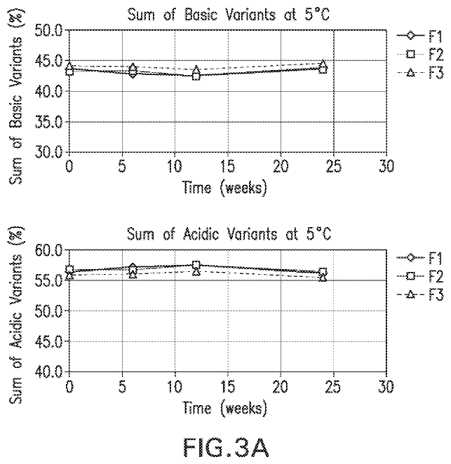

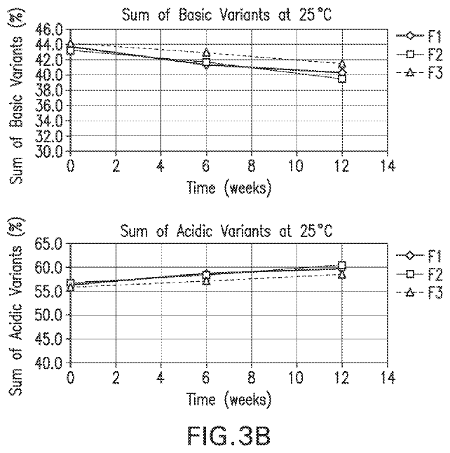

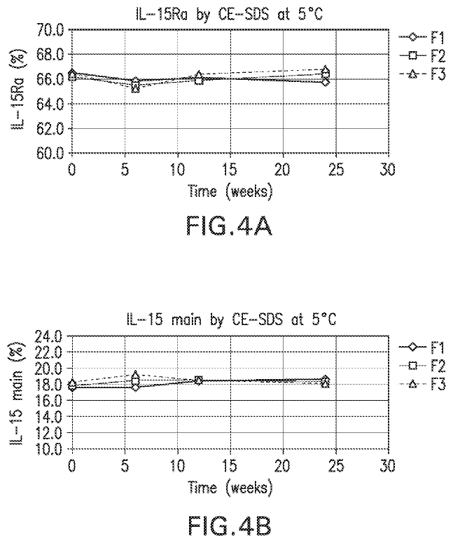

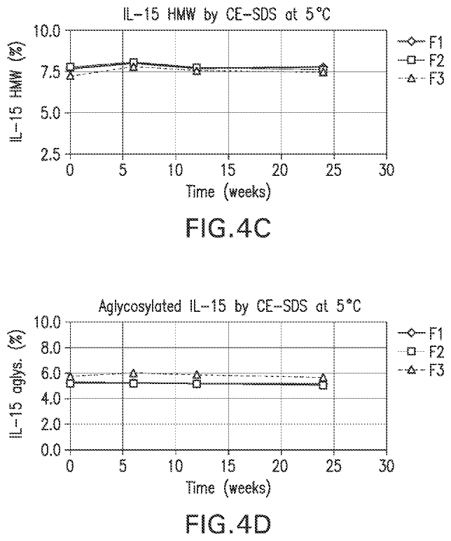

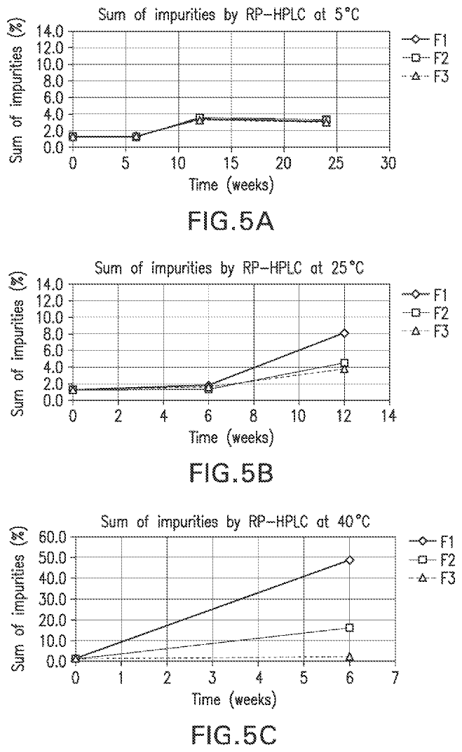

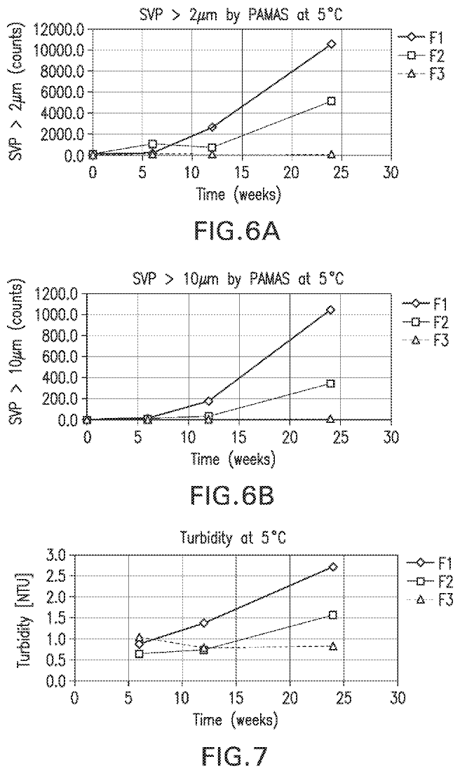

FIG. 3) as well as for purity by CE-SDS (see FIG. 4). However, F3 showed superior stability in contrast to F2 and F1 for accelerated and stressed temperatures when analyzed via RP-HPLC (see FIG. 5). A substantial increase of subvisible particles >2 μm as well as particles >10 μm was observed for F1 and F2 for storage at 2-8° C., while no subvisible particles were observed for F3 (see FIG. 6). These findings were supported by an increase of turbidity for F1 and F2 as shown in FIG. 7.

### Mechanical Stress Testing

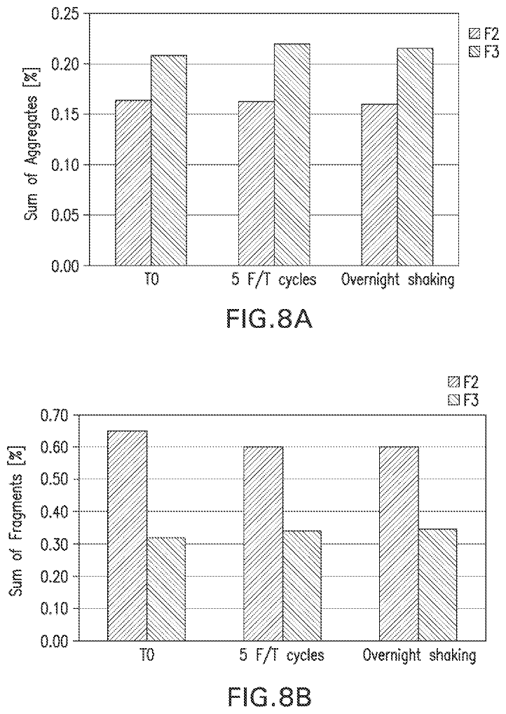

Mechanical Stress testing was performed for F2 and F3 by means of freeze/thaw (F/T) stress and overnight shaking. For testing purposes glass vials were filled with 1.2 mL and subjected to a total of 5 freeze-thaw cycles by iteratively deep freezing the vials in a −80° C. freezer followed by thawing at room temperature. For shaking, the vials were horizontally placed on a shaker and shaken overnight under normal light. All samples were analyzed by SEC. Results are shown in FIG. 8. Both formulations showed good mechanical stability, i.e. no change in aggregates or fragments after F/T or shaking stress.

### EXAMPLE 2

The aim of this study was assess the stability of a liquid pharmaceutical composition comprising heterodimeric IL-15/IL-15Rα complex with IL-15 comprising SEQ ID NO:2 and IL-15Rα comprising SEQ ID NO:5 in a full factorial design to identify the stability contributing factors. 12 different formulations were tested (see Table 5).

TABLE 5
Target composition

| | hetIL-15 | | | Buffer | | Stabilizer | | |
|---|---|---|---|---|---|---|---|---|
| | conc. | | | Conc. | | conc. | | Surfactant |
| ID | (mg/mL) | pH | Buffer | (mM) | Stabilizer | (mM) | Surfactant | concentration |
| F1 | 1 | 4.7 | Acetate | 20 | Sucrose | 260 | Poloxamer 188 | 0.2% |
| F2 | 1 | 4.7 | Acetate | 20 | Sucrose | 260 | Polysorbate 20 | 0.04% |
| F3 | 1 | 5 | Acetate | 20 | Sucrose | 260 | Poloxamer 188 | 0.2% |
| F4 | 1 | 5 | Acetate | 20 | Sucrose | 260 | Polysorbate 20 | 0.04% |
| F5 | 1 | 5.5 | Acetate | 20 | Sucrose | 260 | Poloxamer 188 | 0.2% |
| F6 | 1 | 5.5 | Acetate | 20 | Sucrose | 260 | Polysorbate 20 | 0.04% |
| F7 | 1 | 4.7 | Histidine | 20 | Sucrose | 260 | Poloxamer 188 | 0.2% |
| F8 | 1 | 4.7 | Histidine | 20 | Sucrose | 260 | Polysorbate 20 | 0.04% |
| F9 | 1 | 5 | Histidine | 20 | Sucrose | 260 | Poloxamer 188 | 0.2% |
| F10 | 1 | 5 | Histidine | 20 | Sucrose | 260 | Polysorbate 20 | 0.04% |
| F11 | 1 | 5.5 | Histidine | 20 | Sucrose | 260 | Poloxamer 188 | 0.2% |
| F12 | 1 | 5.5 | Histidine | 20 | Sucrose | 260 | Polysorbate 20 | 0.04% |

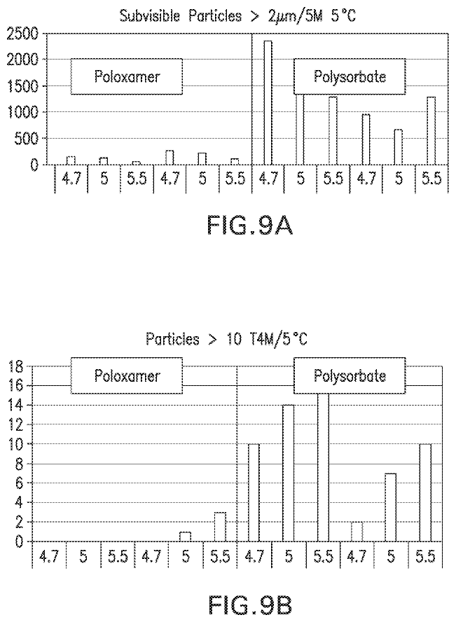

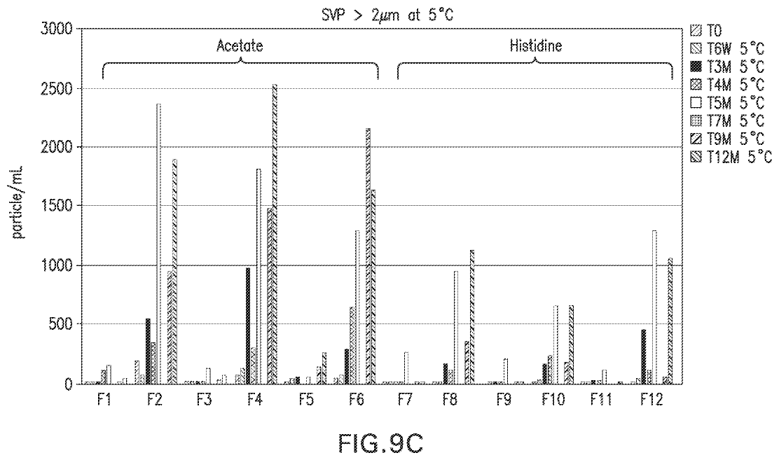

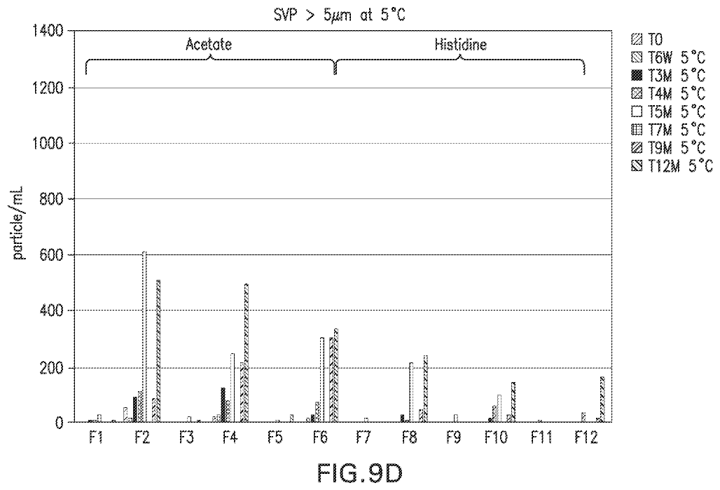

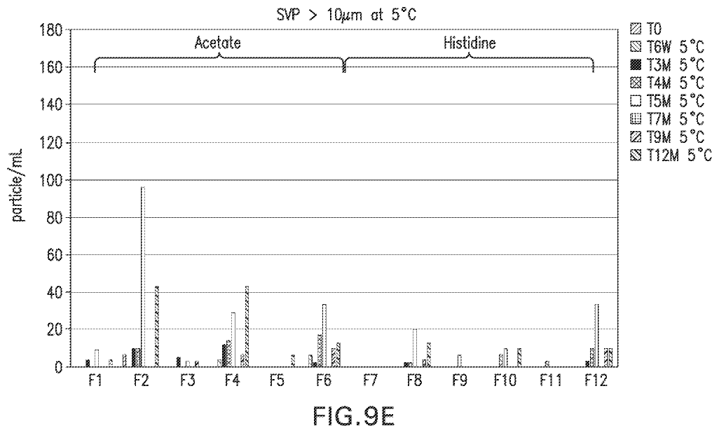

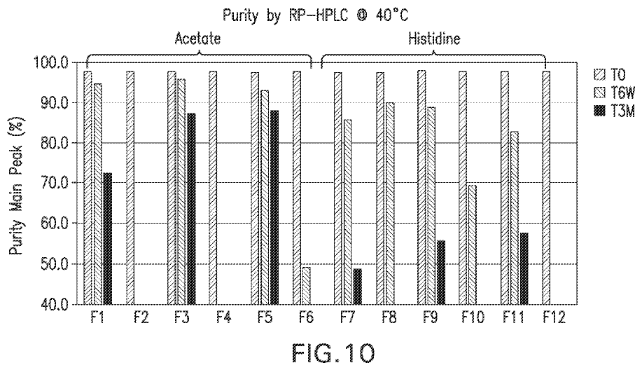

Purity by RP-HPLC and particle formation were assessed for all formulations described above. Decrease of purity was observed by RP-HPLC for all formulations containing polysorbate 20 stored under stressed conditions at 40° C. In contrast, only small adecrease of purity was observed by RP-HPLC for formulations containing poloxamer 188 at 40° C. This observation was less pronounced for formulations containing acetate and more pronounced with formulations containing histidine (see FIG. 10). Formation of subvisible particles (SVP) of size >2 μm (see FIG. 9A) and >10 μm (see FIG. 9B) in the presence of surfactant stored at 2-8° C. was slightly more pronounced for compositions comprising polysorbate 20 while significantly less formation of subvisible particles was observed in presence of poloxamer 188. This increase of particles was less pronounced with formulations containing Histidine than acetate (see FIG. 9C, FIG. 9D and FIG. 9E) but remains within acceptable limits in both cases.

1. A liquid pharmaceutical composition comprising an IL-15/IL-15Rα complex and about 0.0001% to about 1% (w/v) of a surfactant.

2. The composition according to claim 1, further comprising about 1 mM to about 100 mM of a buffering agent providing a pH in the range of from about 4.5 to about 8.5.

3. The composition according to claim 2, further comprising about 1 mM to about 500 mM of at least one stabilizer.

4. The composition according to claim 1, wherein the surfactant is poloxamer.

5. The composition according to claim 4, wherein poloxamer is poloxamer 188.

6. The composition according to claim 4, wherein poloxamer is present at a concentration of about 0.05% to about 0.5% (w/v).

7. The composition according to claim 1, wherein the surfactant is polysorbate.

8. The composition according to claim 7, wherein the polysorbate is polysorbate 20 or polysorbate 80.

9. The composition according to claim 7, wherein the polysorbate is polysorbate 20.

10. The composition according to claim 7, wherein the polysorbate is polysorbate 80.

11. The composition according to claim 7, wherein the surfactant is at a concentration of about 0.01% to about 0.1% (w/v).

12. The composition according to claim 2, wherein the buffering agent is acetate buffer, succinate buffer, citrate buffer or histidine buffer.

13. The composition according to claim 2, wherein the buffering agent is acetate buffer.

14. The composition according to claim 13, wherein the acetate buffer is Na-acetate buffer.

15. The composition according claim 2, wherein the buffering agent is at a concentration of about 10 mM to about 50 mM.

16. The composition according to claim 2, wherein the buffering agent is at a concentration of about 15 mM to about 30 mM.

17. The composition according to claim 2, wherein the buffering agent is at a concentration of about 20 mM.

18. The composition according to claim 2, wherein the buffering agent provides a pH of about 4.7 to about 5.5.

19. The composition according to claim 3, wherein the at least one stabilizer is polyol or sugar.

20. The composition according to claim 3, wherein the at least one stabilizer is a sugar that is sucrose.

21. The composition according to claim 3, wherein at least one stabilizer is present at a concentration of about 100 mM to about 350 mM.

22. The composition according to claim 3, wherein at least one stabilizer is present at a concentration of about 220 mM to about 300 mM.

23. The composition according to claim 3, wherein at least one stabilizer is present at a concentration of about 260 mM.

24. The composition according to claim 1 wherein the concentration of the IL-15/IL-15Rα complex is about 0.1 mg/mL to about 50 mg/mL.

25. The composition according to claim 1, wherein the concentration of the IL-15/IL-15Rα complex is about 0.1 mg/mL to about 10 mg/mL.

26. The composition according to claim 1, comprising about 1 mg/mL of IL-15/IL-15Rα complex, about 0.2% Poloxamer 188, about 260 mM Sucrose, about 20 mM Na-Acetate, and a pH of about 5.0.

27. A solid pharmaceutical composition comprising an IL-15/IL-15Rα complex and about 10 mM to about 50 mM of a buffering agent providing a pH in the range of from about 6.5 to about 8.5, about 1 mM to about 500 mM of at least one stabilizer and about 0.1 mM to about 50 mM of at least one tonicity agent.

28. The composition according to claim 27, wherein the buffering agent is phosphate buffer, acetate buffer, succinate buffer, citrate buffer or histidine buffer.

29. The composition according to claim 27, wherein the buffering agent is Na/K phosphate buffer.

30. The composition according to claim 27, comprising about 1 mM to about 50 mM buffering agent.

31. The composition according to claim 27, comprising about 1 mM to about 5 mM buffering agent.

32. The composition according to claim 27, wherein the pH of the composition is about 6.5 to about 7.5

33. The composition according to claim 27, wherein the pH of the composition is about 7.3.

34. The composition according to claim 27, further comprising about 1 mM to about 500 mM of at least two stabilizers.

35. The composition according to claim 27, comprising about 1 mM to about 500 mM sucrose and about 1 mM to about 500 mM mannitol.

36. The composition according to claim 27, comprising about 5 mM to about 50 mM sucrose and about 100 mM to about 300 mM mannitol.

37. The composition according to claim 27, comprising about 30 mM sucrose and about 220 mM mannitol.

38. The composition according to claim 27, further comprising about 0.1 mM to about 50 mM of at least two tonicity agents.

39. The composition according to claim 27, comprising about 0.1 mM to about 50 mM KCl and about 0.1 mM to about 50 mM NaCl.

40. The composition according to claim 27, comprising about 0.1 mM to about 1 mM KCl and about 10 mM to about 50 mM NaCl.

41. The composition according to claim 27, comprising about 0.375 mM KCl and about 20 mM NaCl.

42. The composition according to claim 27, comprising about 0.1 mg/mL to about 50 mg/mL of IL-15/IL-15Rα complex.

43. The composition according to claim 27, comprising about 0.1 mg/mL to about 10 mg/mL of IL-15/IL-15Rα complex.

44. The composition according to claim 27, comprising about 0.1 mg/mL to about 0.5 mg/mL of IL-15/IL-15Rα complex.

45. The composition according to claim 27, wherein the composition is lyophilized.

46. The composition according to claim 27, comprising about 0.24 mg/mL IL-15/IL-15Rα complex, about 30 mM sucrose, about 220 mM mannitol, about 0.375 mM potassium chloride, about 20 mM NaCl, about 1.35 mM Na/K phosphate buffer at pH of about 7.3.

47. The composition according to claim 27, wherein the IL-15/IL-15Rα complex comprises IL-15 comprising SEQ ID NO: 2.

48. The composition according to claim 27, wherein the IL-15/IL-15Rα complex comprises IL-15Rα comprising SEQ ID NO: 5.

49. The composition according to claim 27, wherein the IL-15/IL-15Rα complex comprises IL-15 comprising SEQ ID NO: 2 and IL-15Rα comprising SEQ ID NO: 5.

50. The composition according to claim 1 for use in the treatment of cancer, lymphopenia, immunodeficiencies, infectious diseases, and/or wounds.

51. The composition according to claim 50 for use in the treatment of cancer.

52. The composition according to claim 50, wherein the cancer is bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodysplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g. mesothelioma), and combinations of said cancers.

53. The composition according to claim 50, wherein the cancer is melanoma, renal cancer, colon cancer, or prostate cancer.

54. The composition according to claim 50, wherein the cancer is melanoma.

55. The composition according to claim 50, wherein cancer is metastatic.

56. A dosage form comprising the pharmaceutical composition of claim 1.

57. A vial comprising the pharmaceutical composition according to claim 1.

58. A syringe comprising the pharmaceutical composition according to claim 1.

59. An autoinjector comprising the syringe of claim 58.

60. An autoinjector comprising the composition according to claim 1.

61. The composition according to claim 1, wherein the liquid composition maintains:
a. about 0.1% to about 0.25% sum of aggregates upon storage at 2-8° C. for 24 weeks;
b. about 0.15% to about 0.35% sum of aggregates upon storage at 25 ° C. for 12 weeks;
c. about 0.3% to about 0.6% sum of aggregates upon storage at 40° C. for 6 weeks;
d. about 0.25% to about 0.75% sum of fragments upon storage at 2-8° C. for 24 weeks;
e. about 1.5% to about 2.0% sum of fragments upon storage at 25 ° C. for 12 weeks;
f. about 2.5% to about 3.5% sum of fragments upon storage at 40° C. for 6 weeks;
g. about 42.5% to about 45% sum of basic variants upon storage at 2-8° C. for 24 weeks;
h, about 55% to about 57.5% sum of acid variants storage at 2-8° C. for 24 weeks;
i about 38% to about 42% sum of basic variants upon storage at 25° C. for 12 weeks;
j. about 55% to about 60% of acidic variants upon storage at 25° C. for 12 weeks;
k. about 65% to about 67% of IL-15Rα by CE-SDS upon storage at 2-8° C. for 24 weeks;
l. about 17% to about 19% of IL-15 by CE-SDS upon storage at 2-8° C. for 24 weeks;
m. about 7% to about 8% of IL-15 HMW by CE-SDS upon storage at 2-8° C. for 24 weeks;
n. about 5% to about 6% aglycosylated IL-15 by CE-SDS upon storage at 2-8° C. for 24 weeks;
o. about 2% to about 4% sum of impurities by RP-HPLC upon storage at 2-8° C. for 24 weeks;
p. about 3% to about 5% sum of impurities by RP-HPLC upon storage at 25° C. for 12 weeks;
q. below 5% sum of impurities by RP-HPLC upon storage at 40° C. for 6 weeks;
r. substantially no SVP>2μm by PAMAS upon storage at 2-8° C. for 24 weeks;
s. substantially no SVP>10μm by PAMAS upon storage at 2-8° C. for 24 weeks;
t. below 1.0 NTU turbidity upon storage at 2-8° C. for 24 weeks;
u. below 0.25% sum of aggregates assessed by SEC after being subjected to five freeze/thaw cycles or overnight shaking; or
v. below 0.35% sum of fragment assessed by SEC after being subjected to five freeze/thaw cycles or overnight shaking.
